(19) United States
(12) Patent Application Publication
(10) Pub. No.: US 2009/0048656 A1
(43) Pub. Date: Feb. 19, 2009

(54) Delivery Device for Delivering a Self-Expanding Stent

(76) Inventor: Ning Wen, Shanghai (CN)

Correspondence Address: GLOBAL IP SERVICES, 7285 W. Eagle Court, Winton, CA, 95388, US

(21) Appl. No.: 12/093,197

(22) PCT Filed: Nov. 7, 2006
(86) PCT No.: PCT/CN2006/002973

§ 371 Date: May 9, 2008

(30) Foreign Application Priority Data
Nov. 9, 2005 (CN) 200510110144.3
Mar. 14, 2006 (CN) 200610024665.1
Mar. 30, 2006 (CN) 200610025297.2

(51) Int. Cl. A61F 2/84

(52) U.S. Cl. 623/1.12

(57) ABSTRACT

The present invention provides a delivery device for self-expanding stent. The delivery device includes pipe head, inner pipe, near end controller, medium pipe, texturing tube, external protection apparatus, at least one locked coil and at least one stayguy. The external protection apparatus is a tearable external protection apparatus or a flexible connects collar hold-down mechanism or a stayguy hold-down mechanism. The present invention has a plenty of following advantages: to locate the stent rotarily, to fix the expanded stent effectively, to reduce the valvular abrasion of artificial cardiac stent, to reduce the abrasion of stayguy, to avoid the dislocation of stayguy and so on.

### FIELD OF THE INVENTION

The present invention provides an delivery device, especially for a self-expanding stent, as a human tissue substitute.

### BACKGROUND OF THE INVENTION

Heart, the most important human organ, is made up left and right parts while each part consists of atria and ventricles. Left and right atria are separated by atrial septum while left and right ventricles are separated by ventricular septum. Four cardiac valves, consisting of tricuspid valve, pulmonary valve, mitral valve and aortic valve, play a crucial role in human blood circulation. The hypoxic blood in the systemic circulation enters the right atrium through vein and the right ventricle through the tricuspid valve in turn. And then the blood is pumped into pulmonary circulation through the pulmonary valve by the right ventricular systole. After the oxygen saturation in the pulmonary circulation, the blood goes back to the left atrium through vein and reaches the left ventricle through mitral valve. In the end, the blood is pumped into the aorta through aortic valve by left ventricular systole and returns to the systemic circulation again. Left and right coronary artery openings are located below the aortic valve. The structures of the four cardiac valves ensure the valves open when blood circulation is in right direction, which reduces heart burden caused by blood backstream, otherwise they will close. However, such structures might lead to some acquired injury or pathological changes of the cardiac valves, for various reasons such as rheumatism, atherosclerosis and so on. In addition, there are some congenital heart diseases such as the tetralogy of Fallot whose remote post-operative effect can also generate the pathological changes of the pulmonary valve. The valvular lesion can cause the valves' functions lose gradually. For example, the valvular insufficiency can lead to blood backstream, the narrow valves can bring about difficult blood circulation, or both of the two effects. The process mentioned above will make the heart burden so heavily that it will bring about the exhaustion of heart functions. The traditional treatment to the acquired injury or pathological changes of the cardiac valves is to operate a thoracotomy, which is to open the heart to operate the plastics of the valve lesion or artificial cardiac valve replacement with the support of extracorporeal circulation after the heart ceases beating. Current artificial cardiac valve can be classified as two categories: metal mechanical valve and biologic valve. Biologic valve is from processing animal materials such as bovine pericardium, valved bovine jugular vein and porcine aortic valve. The above-mentioned open-heart surgery is characterized as long operation time, high cost, profound wound and high risk. Furthermore, for one thing, the patients need to take a long time to operate anticoagulation therapy after they perform artificial cardiac valve replacement. For another, because of the limited lifespan of the biologic valve materials, patients often need an extra operation.

In order to solve the defects caused by the thoracotomy, people employ the method of percutaneous intervention to implant artificial cardiac valve instead of attempting to operate an open-heart surgery. Currently, there are two kinds of technologies for the interventional artificial cardiac valve.

1. Balloon Expanding Artificial Cardiac Valve

This kind of balloon expanding artificial cardiac valve is a biologic valve. In order to reach the valve's functional mode, we can adopt such an interventional way that is to set the biologic valve on a plastometric stent respectively and compress the valve on a balloon in a radial direction to minify its diameter, implant percutaneously and press the balloon to expand and set the stent.

2. Self-Expanding Artificial Cardiac Valve

This kind of artificial valve owns an elastic stent which can expand by itself under radial compression.

Drum-type stent in the valve's intermediate section, self-expanding and strengthened man-made stent and conjoined delivery device are mentioned in the invention whose Chinese application number for patent of invention is 200410054347.0.

The disadvantages and problems of the self-expanding artificial valve is the friction force between self-expanding artificial cardiac valve and sheath is so great that it influences the artificial valve's accurate release.

When the stayguy of the conjoined delivery device passes through the deformable unit of prosthetic valve, the friction force is enlarged; while it does not pass through the unit, the stayguy is easy to dislocate its position.

The disadvantages and problems the balloon expanding and self-expanding artificial cardiac valve mentioned above own commonly are as follows:

1. Current delivery device for interventional self-expanding stent and the stent under radial compression are so hard that they lack bending property and can not pass through the aortic arch easily and aim at the natural aortic valve opening.

2. Even with the help of X-ray inspection, interventional self-expanding stent and its delivery device can not be located in the valve's axial upward and backward position easily because the anatomic site can not be judged accurately and the artificial valve become unsteady due to the surging of the blood stream. If the interventional artificial aortic valve locates upward, it will exercise an influence on mitral valve; if it locates backward, it will block the coronary artery opening.

3. The location of the rotation direction of the interventional aortic valve self-expanding stent and its delivery device is not resolved. If the interventional aortic valve rotates in a wrong direction, it will block the coronary artery opening.

4. The retroversion of the sheath will meet with a good deal of resistance if the self-expanding stent is under high compression. The resistance and difficulty of the sheath's retroversion still enable the operator to displace the located self-expanding stent.

5. During its release period, the stent shifts from semi-expanding to fully expanding gradually. The time needed exceeds one heartbeat cycle. The stent's position will be altered due to blood stream surging. On the contrary, the expanding stent will block the blood stream and the balloon self-expanding stent will fully block the blood stream during the balloon expanding process.

Currently, we can employ three different ways to compress the self-expanding stent, self-expanding tectorial stent and self-expanding stent valve in their radial directions to reduce their diameters and then implantation them into the patients' bodies through minimally invasive surgery.

a) sheath;

b) lacerable membrane outside the stent;

c) stent's binding implantation system.

### SUMMARY OF THE INVENTION

The goal of the present invention is to overcome the above-mentioned problems current technologies possess and provide a novel delivery device for self-expanding stent, not only for interventional therapy but also for minimally invasive therapy.

The technical scheme of the present invention is an delivery device for self-expanding stent. The device which includes a pipe head, an inner pipe, a near-end controller, a medium pipe, a texturing tube, an external protection apparatus, at least one locked coil and one stayguy. The above pipe head, inner pipe and near-end controller interconnect in order. The above sleeve pipe of the medium pipe slides along the inner pipe. The above texturing tube is installed in the incorporated pipe head, inner pipe and near-end controller. The above external protection system envelops outside the inner and medium pipe. The above locked coil and stayguy thread through the incorporated inner pipe and near-end controller.

The inner pipes in above recited delivery device for self-expanding stent possess a long tube structure. At least one pore space through which various strings and threads pass is installed in the inner pipe and at least one lateral opening is installed in the far-end of the inner pipe.

At least one texturing tube through which various stings and threads pass is installed in the inner pipe in above recited delivery device for self-expanding stent. The texturing pipe can slide along other texturing pipes and inner pipes.

At least one texturing tube, through which various string and threads pass, is installed in the inner pipe in above recited delivery device for self-expanding stent. The texturing pipes are set to affix with other texturing pipes and inner pipes and can not slide along each other.

The plaited strengthening net can be installed in the intermediate layer of the inner pipe in above recited delivery device for self-expanding stent and the lateral opening of the inner pipe can be located in one of the meshes of the plaited strengthening net.

The inner pipe in above recited delivery device for self-expanding stent is a coiled spring tube. The spring wires in the distal segment of the spring tube partially constitute a semi or whole cycle, forming the far lateral opening, intermediolateral opening and proximal opening of the inner pipe.

Polymer material tube is installed outside the above coiled spring tube in above recited delivery device for self-expanding stent. Openings are set on the polymer material tube corresponding to the semi or whole cycle of the spring tube.

The near-end controller in above recited delivery device for self-expanding stent has a dendritic structure. The near-end controller contains at least one main pipe, and at least one branch pipe of the stayguy, one branch pipe of the locked coil, one branch pipe of flushing and angiography, one branch pipe of thread, which are all connected with the main pipe.

The close segment of the medium pipe in above recited delivery device for self-expanding stent connects with a lateral pipe. One stayguy is set in the medium pipe. The far-end of the stayguy is fixed on the far-end port of the medium pipe. The near-end of the stayguy is drawn out of the lateral pipe of the close segment of the medium pipe. The fixed point of the far-end of the stayguy and the lateral pipe of the close segment of the medium pipe are set at the same plane or side. Each port of the near-end of the medium pipe and each port of the lateral pipe have a shrink-ring, which can slide along the inner pipe when loosened and can fix on certain position of the inner pipe when tightened.

The external protection apparatus in above recited delivery device for self-expanding stent is tearable. The external protection apparatus contains a tearable sheath, a stayguy applied to frap the tearable sheath temporarily, a locked coil applied to lock the stayguy of the sheath. A lengthwise opening throughout the far-end is set on the distal segment of the tearable sheath. Several take-up openings are set on the bilateral lengthwise openings. The structure of the above tearable sheath is a tubular structure of tube layout or mesh duct.

The external protection apparatus in above recited delivery device for self-expanding stent is a take-up and hold-down mechanism, which contains at least one locked coil, at least one connect collar linking to the stent, at least one take-up wire used for compressing the stent. A take-up collar is set on the far-end of the take-up wire. After it is locked at its far-end by the take-up collar, the take-up wire passes through the lateral opening of the inner pipe and wraps between the connect collar on the locked coil, stent and the outboard stent, forming a hold-down mechanism which can be locked or unlocked as the case might be. The near-end of the take-up wire is drawn out of the near-end of the implantation system and is fixed to the near-end controller of the implantation system temporarily.

One take-up wire and one connect collar are installed in above recited delivery device for self-expanding stent. The connect collar is a closed line eye or a closed flexible connect collar linking to the stent. Locked at its far-end cycle by the locked coil, the take-up wire passes through the closed line eye or the closed flexible connect collar on the same section of the stent and rounds the locked coil in the inner pipe to form a semi-cycle of take-up wire. In the end, it circles outside the stent once and wraps to form a hold-down mechanism which can be locked or unlocked as it depends at a certain section of the stent.

One take-up wire and one connect collar are installed in above recited delivery device for self-expanding stent. The connect collar is a closed line eye or a closed flexible connect collar linking to the stent. Locked at its far-end cycle by the locked coil, the take-up wire passes through the closed line eye or the closed flexible connect collar on the same section of the stent and rounds the locked coil in the inner pipe to form a semi-cycle of take-up wire. In the end, it circles outside the stent twice in both directions and wraps to form a hold-down mechanism which can be locked or unlocked as it might be at a certain section of the stent.

One take-up wire and two connect collars are installed in above recited delivery device for self-expanding stent. The two connect collars are provided by a hold-down mechanism of flexible connect collar on the stent. The hold-down mechanism of flexible connect collar circles the stent under radial compression in less than one lap, forming two opposite free cycles as the connect collars. Locked at its far-end cycle by the locked coil, the take-up wire passes through the two free cycles of the hold-down mechanism of the flexible connect collar and rounds the locked coil in the inner pipe to form a semi-cycle of the take-up wire, forming a hold-down mechanism which can be locked or unlocked as it depends at a certain section of the stent.

One take-up wire and several connect collars are installed in above recited delivery device for self-expanding stent. Several connect collars are provided by a hold-down mechanism of flexible connect collar on the stent. The hold-down mechanism of flexible connect collar circles the stent under radial compression in less than one lap, forming two opposite free cycles as the connect collars. Locked at its far-end cycle by the locked coil, the take-up wire passes through the two opposite free cycles of the hold-down mechanism of the closed line eye or the flexible connect collar at different sections of the stent one after another and carries out lockable and undone circles, forming a continuous multi-sectional hold-down mechanism.

One take-up wire and several connect collars are installed in above recited delivery device for self-expanding stent. Several connect collars are provided by a hold-down mechanism of flexible connect collar on the multi-sectional stent. Every hold-down mechanism of the flexible connect collar circles the stent under radial compression in less than one lap, forming two opposite free cycles as the connect collars. Locked at its far-end cycle by the locked coil, the take-up wire wraps the stent at different lockable and undone sections in one direction and comes back along the same course to warp the lockable and undone stent for the second time, forming a continuous multi-sectional hold-down mechanism. In the process of the sintering, the take-up wire rounds the locked coil in the inner pipe to form a semi-cycle of the take-up wire.

Two take-up wires and several connect collars are installed in above recited delivery device for self-expanding stent. Several connect collars are provided by a hold-down mechanism of the flexible connect collar on the multi-sectional stent. Every hold-down mechanism of the flexible connect collar circles the stent under radial compression in less than one lap, forming two opposite free cycles as the connect collars. Locked at their far-end cycles by the locked coil, the two take-up wires wrap the stent at different lockable and undone sections in opposite symmetrical directions, forming a continuous multi-sectional hold-down mechanism. In the process of the sintering, two take-up wires round the locked coil in the inner pipe, forming two semi-cycles of the take-up wires, respectively.

Two take-up wires and several connect collars are installed in above recited delivery device for self-expanding stent. Several connect collars are provided by a hold-down mechanism of the flexible connect collar on the multi-sectional stent. Every hold-down mechanism of the flexible connect collar circles the stent under radial compression in less than one lap, forming two opposite free cycles as the connect collars. Locked at their far-end cycles by the locked coil, the two take-up wires wrap the stent at different sections lockably and unlockably in one of the opposite symmetrical directions and come back along the same course to wrap the stent lockably and unlockably for the second time. In the end, they form a continuous multi-sectional hold-down mechanism. In the process of the sintering, two take-up wires round the locked coil in the inner pipe to form semi-cycles of the take-up wire, respectively.

The hold-down mechanism of the flexible connect collar in above recited delivery device for self-expanding stent is a flexible connect collar with two free cycles whose fixed point circles and ties to a cross-point of mesh wires. Two free cycles extend bilaterally along the outside superficial circularity of the stent and circle the stent under radial compression in less than one lap to form an opposite condition.

The hold-down mechanism of the flexible connect collar in above recited delivery device for self-expanding stent contains several flexible connect collars. The fixed points of two flexible connect collars among them circle and tie to the same cross-point of the mesh wires. The fixed ends of the other flexible connect collars circle and tie to other cross-points of the mesh wires in the same circumference of the above cross-point. The free cycle of every flexible connect collar extends bilaterally along the outside superficial circularity of the stent and circle the stent under radial compression in less than one lap. Extending in the same direction, the free cycles are connected one by one in order. Extending in two directions, the last two free cycles form an opposite condition.

The hold-down mechanism of the flexible connect collar in above recited delivery device for self-expanding stent also contains temporary stayguys. Locked temporarily at its far-end cycle by the locked coil, the take-up wire passes through a closed line eye on the stent and then circles the surface of the stent under radial compression close to a cycle and comes back to the same closed line eye again. Afterwards, it passes through the interlayer between the inner pipe and the medium pipe and is drawn out of the near-end of the implantation system. The temporary stayguy is set between the inner pipe and the medium pipe. The temporary stayguy rounds the take-up wire near the far-end of its cycle to form double lines and holds the take-up wire to the near-end.

The external protection apparatus in above recited delivery device for self-expanding stent is a hold-down mechanism of the flexible connect collar, which contains at least one locked coil, one flexible connect collar linked to the stent and applied to compress the stent, one flexible connect collar passed through the lateral opening of the inner pipe and locked by the temporarily passing locked coil.

The flexible connect collar in above recited delivery device for self-expanding stent contains fixed points and free points. The fixed point circles and ties to the arched inflexion or the closed line eye or the cross-point of the mesh wires of the stent. The free point extends inside or outside the stent and composes the single free cycle or double free cycle.

The flexible connect collar in above recited delivery device for self-expanding stent is a single closed wire loop, which passes through the arched inflexion or the closed line eye or the cross-point of the mesh wires of the stent. It can slide but can not break away.

The hold-down mechanism of the flexible connect collar in above recited delivery device for self-expanding stent contains several flexible connect collars with a single free cycle. The flexible connect collars are distributed circularly on the same section inside or outside the stent. The adjacent flexible connect collars under the radial compressed stent are connected one by one in order and circle the stent once. The last free cycle of the flexible connect collar passes through the lateral opening of the inner pipe and is locked by the temporarily passing locked coil.

The hold-down mechanism of the flexible connect collar in above recited delivery device for self-expanding stent contains only one flexible connect collar with a single free cycle. The flexible connect collar under the radial compressed stent circles outside the stent once, passes through the lateral opening of the inner pipe, and then is locked by the temporarily passing locked coil.

The hold-down mechanism of the flexible connect collar in above recited delivery device for self-expanding stent contains only one flexible connect collar with a double free cycle. The two free cycles of the flexible connect collar circle the radial compressed stent in opposite directions half cycle, respectively. They pass through the same lateral opening of the inner pipe and are locked by the temporarily passing locked coil.

The hold-down mechanism of the flexible connect collar in above recited delivery device for self-expanding stent contains several flexible connect collars with a single free cycle. The flexible connect collars are distributed helically outside the stent. The adjacent flexible connect collars under the radial compression of the stent are connected one by one in order and circle the stent at least once. The last free cycle of the flexible connect collar passes through the lateral opening of the inner pipe and is locked by the temporarily passing locked coil.

The hold-down mechanism of the flexible connect collar in above recited delivery device for self-expanding stent contains one flexible connect collar with a double free cycle and several flexible connect collars with a single free cycle. The flexible connect collars are distributed helically outside the stent. One flexible connect collar with a double free cycle is set up in the middle of the stent. Two free cycles of the flexible connect collar extend in opposite directions under the radial compression of the stent and are connected with the adjacent flexible connect collar with a single free cycle one by one in order at least once. The last two free cycles extending in opposite directions of the flexible connect collar pass through the far lateral opening and the proximal opening of the inner pipe respectively and are locked by the temporarily passing locked coil.

The hold-down mechanism of the flexible connect collar in above recited delivery device for self-expanding stent also contains temporary stayguys which make up of the implantation system. The flexible connect collar applied to compress the stent is a single closed wire loop. One port passes through the arched inflexion or the closed line eye or the cross-point of the mesh wires of the stent. Another one passes through the lateral opening of the stent and the inner pipe to enter the inner pipe, or enters between the inner pipe and the medium pipe and is locked by the temporarily passing locked coil. The middle one is pulled by the temporary stayguys.

The closed wire loop in above recited delivery device for self-expanding stent is passed by a stayguy and can be pulled to the near-end. A cycle is set up at the far-end of the stayguy and is locked by the temporarily passing locked coil of the inner pipe or the locked coil between the inner pipe and the medium pipe.

At least one lateral texturing tube is installed in above recited delivery device for self-expanding stent. Beginning from the outside of the medium section to the distal segment of the inner pipe, the lateral texturing tube is connected with the distal segment of the inner pipe. Extending to the medium segment of the inner pipe along the proximal direction of the inner pipe, the lateral texturing tube can also extend to the close segment of the inner pipe or near-end controller. Located between the far lateral opening and the proximal opening of the inner pipe, the far-end of the lateral texturing tube bends outward to form a certain angle between the direction of the port and the direction of the lateral opening of the inner pipe.

One lateral texturing tube in above recited delivery device for self-expanding stent is set up coordinating with the external protection apparatus.

Two lateral texturing tubes in above recited delivery device for self-expanding stent are set up coordinating with the hold-down mechanism of the take-up wire or the hold-down mechanism of the flexible connect collar.

The locked coil in above recited delivery device for self-expanding stent passes through the inner pipe. Its far-end passes through the cycle of one or more stayguys and locks one or more stayguys. Its near-end is connected with the sliding-jig in the branch pipe of the locked coil of the near-end controller.

The stayguy in above recited delivery device for self-expanding stent passes through the pore space of the inner pipe. Its far-end has a cycle of stayguy and its near-end is drawn out of the branch pipe of the stayguy of the near-end controller. Its distal segment is drawn out of the lateral opening of the inner pipe, forming the outside segment of the stayguy. The outside segment of the stayguy circles the net stent once, enters the same lateral opening of the inner pipe and then is locked through its far-end cycle of the stayguy by the locked coil.

The outside segment of the stayguy in above recited delivery device for self-expanding stent passes through the opening of the deformable unit or the arched inflexion or the closed line eye and the flexible connect collar to form a lasso when it circles the stent.

The above recited delivery device for self-expanding stent also contains the B-mode ultrasonic probe, which is set up on the back-end of the pipe head or near the far lateral opening or proximal opening of the inner pipe. Being connected with the B-mode ultrasonic probe, the conduit passes through the inner pipe and is drawn out of the near-end controller.

The above recited delivery device for self-expanding stent also contains the temporary recovering wire, which is applied to assist the stayguy come back to the near-end controller when the device is assembled.

### BRIEF DESCRIPTION OF THE DRAWINGS

Through the following description of several instances combined with figures of the artificial cardiac stent and its delivery device in present invention, we can understand the goal, detailed structural characteristics and merits of the present invention further. The figures are:

FIG. 1a is the front view of the delivery device for a self-expanding stent in the present invention;

FIG. 1b is the structural representation of sectional view for FIG. 1a;

FIG. 1c is the enlarged sectional view of part B in FIG. 1b;

FIG. 1d is the structural representation of sectional view for the near-end controller of the delivery device in FIG. 1a;

FIG. 2a, FIG. 2b, FIG. 2c and FIG. 2d are the structural representations of sectional view along Line AA for the inner pipe of the delivery device for a self-expanding stent as shown in FIG. 1a;

FIG. 2e is the partial side view of distal segment for the inner pipe with spring type in present delivery device for a self-expanding stent;

FIG. 3a is the structural representation of sectional plan view for the medium pipe in present delivery device for a self-expanding stent;

FIG. 3b is the structural representation of sectional plan view for the outer sheath in present delivery device for a self-expanding stent;

FIG. 4a is the front view of the monolayer stent of the artificial cardiac stent under radial compression in present invention;

FIG. 4b and FIG. 4c are the front views of the stent with outer free tongue of the intermediate section of the artificial cardiac stent in present invention. Among them,

FIG. 4b shows the fully radial compression of the stent and FIG. 4c shows the release and expansion of the outer tongue of the compressed stent;

FIG. 4d is the view towards C-C of the FIG. 4a;

FIG. 4e is the vertical view of FIG. 4b after the transecting of the inner pipe's intermediate section;

FIG. 4f is the vertical view of FIG. 4c after the transecting of the inner pipe's intermediate section;

FIG. 5a, FIG. 5b, FIG. 5c and FIG. 5d are the 3D side views of related structural parts of the inner pipe's distal segment in present delivery device for a self-expanding stent. Among them, the stents in FIG. 5a, FIG. 5b and FIG. 5d only are shown in the far-end and near-end. FIG. 5a indicates one locked coil and three stayguys. FIG. 5b shows two locked coils, one controls the far and close two stayguys in turn, another controls the medium stayguy only. FIG. 5c and FIG. 5d show the assembling process of the stent. Among them, FIG. 5c shows the withdrawing line for assembling and the locked stayguy, but no stent, FIG. 5d shows the assembling process of the stent:
the far-end of the stent: the stayguy is going to pass through the closed line eye and the flexible connect collar;
the middle part of the stent: the stayguy has passed through the closed line eye and the flexible connect collar; recover the inner side of the stent again and is going to be withdrawn by the temporary withdrawing line of the stayguy's close segment outside the near-end controller;
the near-end of the stent: the stayguy has been withdrawn outside the near-end controller by the withdrawing line for assembling;

FIG. 6a, FIG. 6b and FIG. 6c are the side views of distal segment for the inner pipe with a side texturing tube. Among them, FIG. 6a and FIG. 6b show the tearable sheath with tubular framework of tube layout outside the inner pipe and stent, and withdrawing line of the sheath at the openings; FIG. 6c shows the tearable sheath with tubular framework of mesh duct outside the inner pipe and stent, and withdrawing line of the sheath at the openings;

FIG. 7 is the side view of distal segment for the inner pipe with two side texturing tubes in present delivery device for a self-expanding stent, and outside the stent there are sintered and unhitched pressing wires;

FIG. 8 is the first instance of the flexible connect collar in the hold-down mechanism and the structural representation of cross section for the related connecting pieces in present invention;

FIG. 9 is the second instance of the flexible connect collar in the hold-down mechanism and the structural representation of cross section for the related connecting pieces in present invention;

FIG. 10 is the third instance of the flexible connect collar in the hold-down mechanism and the structural representation of side view for the related connecting pieces in present invention;

FIG. 11 is the fourth instance of the flexible connect collar in the hold-down mechanism and the structural representation of side view for the related connecting pieces in present invention;

FIG. 12 is the fifth instance of the flexible connect collar in the hold-down mechanism and the structural representation of the related connecting pieces (partially showing the double layer of inside and outside) in present invention;

FIG. 13 is the sixth instance of the flexible connect collar in the hold-down mechanism and the structural representation of the related connecting pieces in present invention;

FIG. 14 is the first instance of the stayguy in the hold-down mechanism and the stereo structural representation of the related connecting pieces in present invention;

FIG. 15 is the second instance of the stayguy in the hold-down mechanism and the stereo structural representation of the related connecting pieces in present invention;

FIG. 16 is the third instance of the stayguy in the hold-down mechanism and the stereo structural representation of the related connecting pieces in present invention;

FIG. 17 is the fourth instance of the stayguy in the hold-down mechanism and the stereo structural representation of the related connecting pieces in present invention;

FIG. 18 is the fifth instance of the stayguy in the hold-down mechanism and the structural representation of the front view of the related connecting pieces in present invention;

FIG. 19 is the sixth instance of the stayguy in the hold-down mechanism and the structural representation of the front view of the related connecting pieces in present invention;

FIG. 20 is the seventh instance of the stayguy in the hold-down mechanism and the stereo structural representation of the related connecting pieces in present invention.

### DETAIL DESCRIPTION OF THE INVENTION

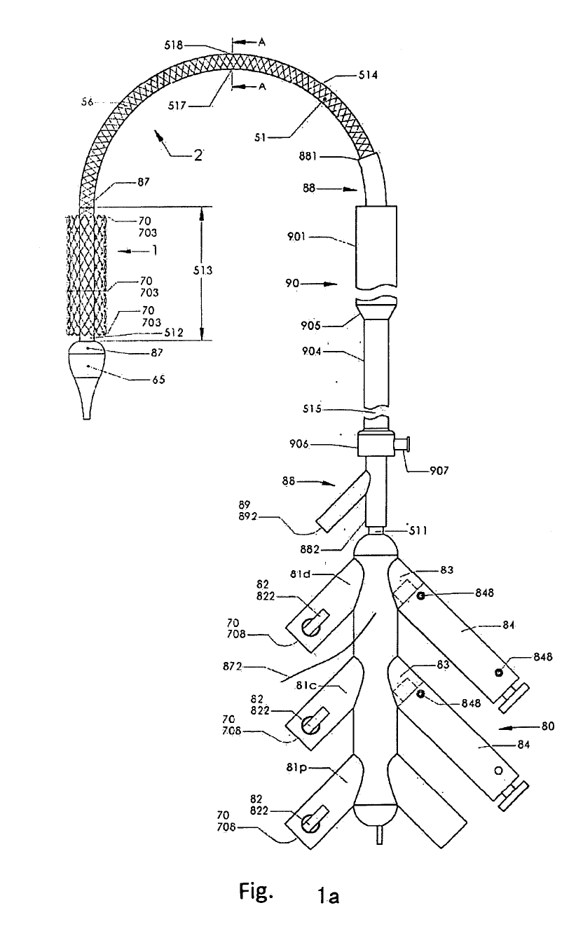

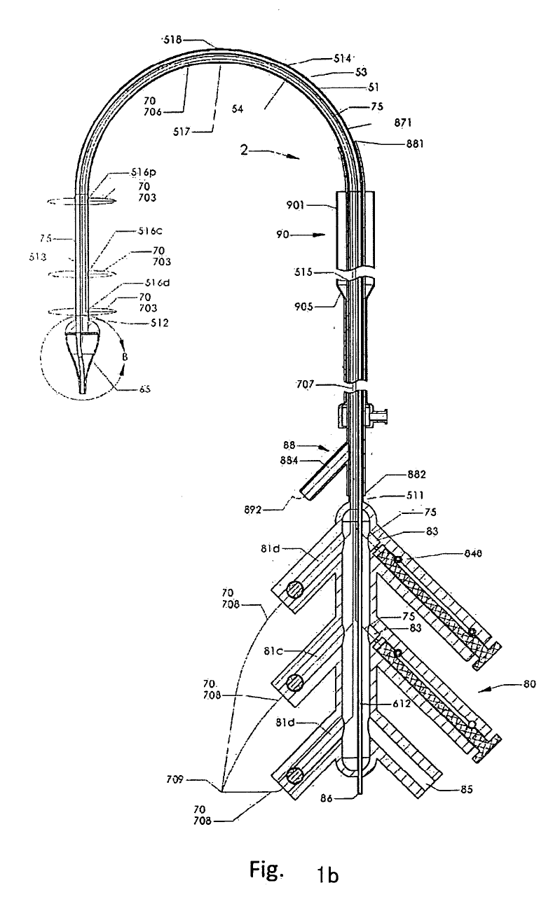

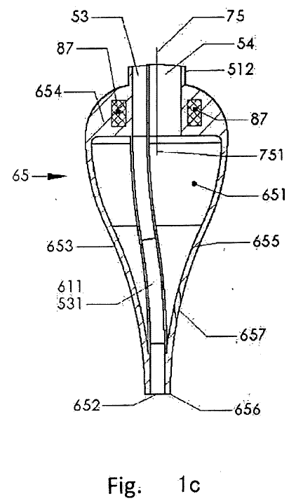

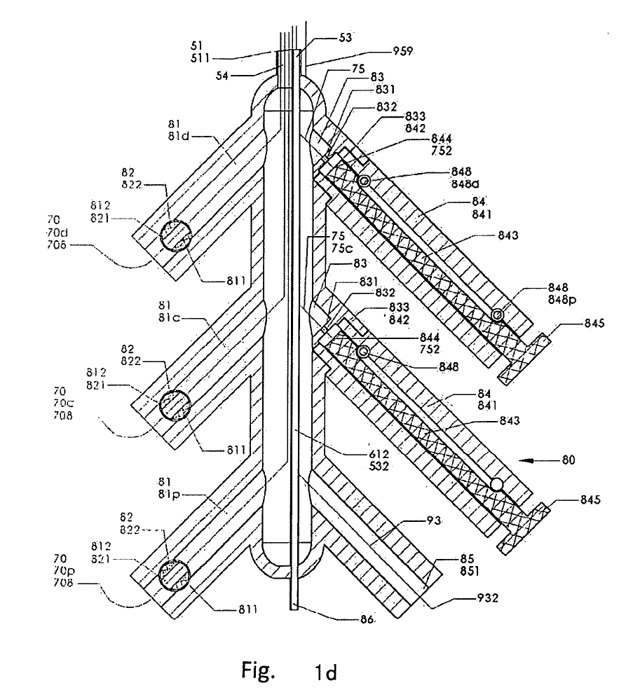

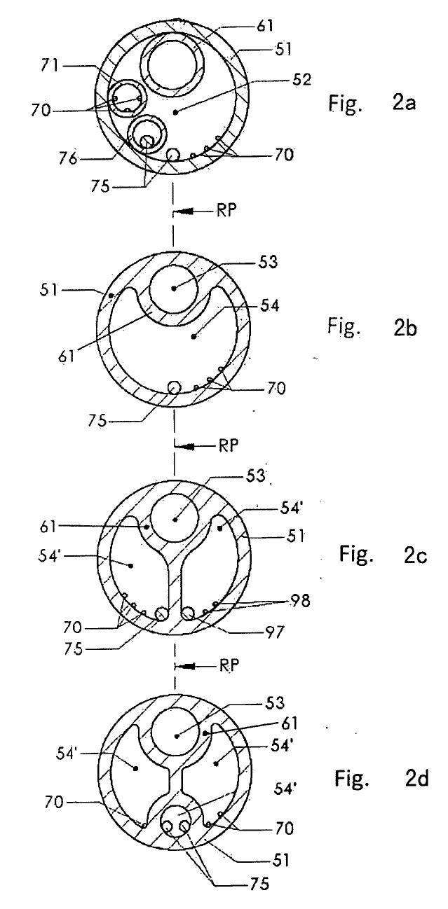

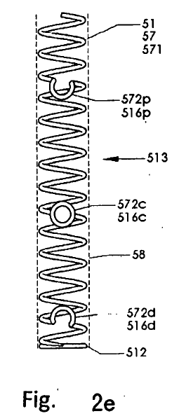

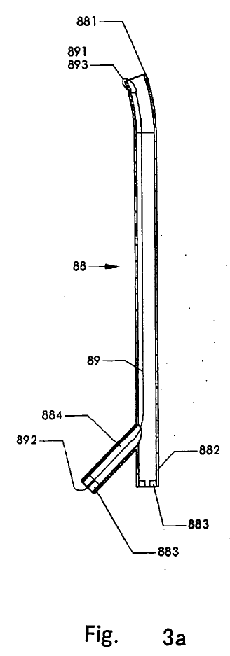

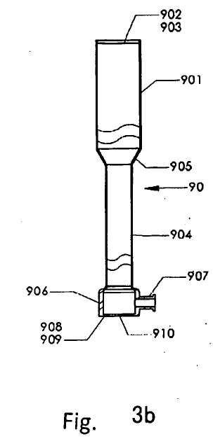

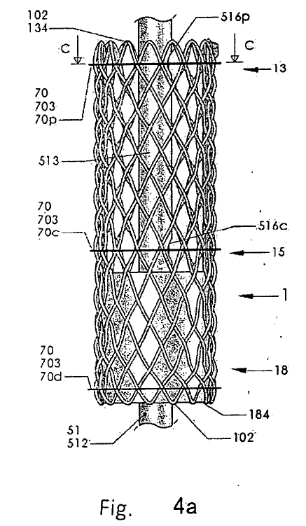

Referring from FIG. 1 to FIG. 7, the delivery device (2) for the self-expanding stent (1) in present invention include not only the inner pipe (51), the texturing tube (61), the pipe head (65), the stayguy (70), the locked coil (75), the near-end controller (80), the medium pipe (88), the external protection apparatus (90, 92, 96), but also the B-mode ultrasonic probe (87) and the side texturing tube (99).

Referring to FIG. 1a, combining with FIG. 2a, FIG. 2b, FIG. 2c and FIG. 2d, the inner pipe (51) of the delivery device (2) for the self-expanding stent (1) in present invention has a long tube structure. Its section might be circular. The inner pipe (51) can be made from polymer material with good flexibility and high strength. The transparent or semitransparent inner pipe (51) favors the removing of air bubble before operation. The inner pipe (51) is 80-150 cm long. Its near-end (511) is outside the body while its far-end (512) reaches the natural valves of the heart. The far-end (512) of the inner pipe links with the pipe head (65). The pore space (52, 54, 54′) of the inner pipe is connected with the conical slight lumen (651) of the pipe head (65). The structure of the inner pipe (51) can be different. The inner pipe (51) can possess one or more pore spaces (52, 53, 54, 54′) which are connected with each other.

Referring to FIG. 2a, the structure of the inner pipe (51) can be a monaulic inner pipe (51) with one unique large circular versatile pore space (52). One or more texturing tubes (61), stayguys (70) and locked coils (75) can be located inside the large circular versatile pore space (52). They (61, 70 and 75) can slide within each other. One or more texturing tubes (61), stayguy tubes (71), locked coil tubes (76) can also be located inside the large circular versatile pore space (52). The threads and strings enter relevant tubes and never cross with each other. Many small pipes (61, 71 and 76) in monaulic pipe make up a multiporous pipe and they can slide with each other.

Referring to FIG. 2e, the inner pipe (51) can be composed of helical spring (57). The helical spring (57) can be made up of single thread (571) or numerous threads. The diameter of the spring wire (571) is equivalent to the inner pipe's wall thickness. The inside and outside diameters of the spring wire might be changeable. The far-end of the spring wire is thinner while the far and near ends are thicker. The spring wire (571) can compose the semi-cycle (572d, 572p) or the whole cycle (572c) partially, and is equivalent to the far lateral opening (516d), intermediolateral opening (516p) and proximal opening (516c). The pace of the spring (57) can be different: 1. If it is equal to the diameter of the thread, then the spring can only be extended (called pullback spring); 2. If it is greater than the diameter of the thread, then the spring can be extended or shrinking (called pressure spring). The spring (57) can be protected by the polymer material pipe (58) to reduce the external friction and to avoid leakage. Nothing but at least two ends of the polymer material pipe linking tightly with it can ensure the spring's folding resistance without axial extending.

Referring to FIG. 2b, with two pore spaces (53, 54), the inner pipe (51) can also be biforous. One is a small circular texturing tube 0.035″ (53) providing the passage of thread 0.035″ and equivalent to the texturing tube (61), another is a large semi-lunar ordinary pipe space (54) providing the passage of the stayguy (70), the locked coils (75), the possible stent pressing wire (98) and the locked coils (97). The distribution of the two pipe spaces (53, 54) is non-central and the pipes can not slide as affixing with each other.

Referring to FIG. 2c and FIG. 2d, the structure of the inner pipe (51) can also be multi-porous with many pore spaces (53, 54′). On the basis of the bi-porous pipe, the multi-porous pipe has two or more small pipe spaces (54′) divided from the large semi-lunar ordinary pipe space (54). A small circular texturing tube 0.035″ (53) provides the passage of thread 0.035″, equivalent to the texturing tube (61), and many small pipe spaces (54′) provide the passage of the stayguy (70), the locked coils (75), the possible stent pressing wire (98) and the locked coils (97), respectively. These tubes affix with each other and can not slide.

Referring to FIG. 1a and FIG. 1b, combining with FIG. 5a, FIG. 5b, FIG. 5c, FIG. 5d, FIG. 6a, FIG. 6b, FIG. 6c and FIG. 7, the distal segment of the inner pipe (513) is located nearside the far-end of the inner pipe (512). While assembling and transporting, the distal segment of the inner pipe (513) is located inside the self-expanding stent (1) and is employed to connect the self-expanding stent (1). Its inside diameter is about 1.5-2.5 mm and its outside diameter is about 1.8-3.0 mm. Its length is slightly longer than that of the compressed self-expanding stent (1). The distal segment of the inner pipe (513) has one or more side openings (516d, 516c, 516p) at different distance. The far-side opening (516d), the intermediolateral opening (516c) and the proximal opening (516p) are all on the same plane or same side of the inner pipe, such as all on the concave plane (517). The far-side opening (516d), the intermediolateral opening (516c) and the proximal opening (516p) are relative to the upper end (184), the medium section (15) and the lower end (134), respectively. The distance between the far-side opening (516d) and the proximal opening (516p) is probably equal to the length of the compressed stent (1). The side openings (516d, 516c, 516p) are connected with the large circular versatile pore space (52) of the monaulic inner pipe (51), or connected with the large semi-lunar ordinary pipe space (54) of the biforous inner pipe (51), or connected with the stayguy pipe space, the locked coil pipe space, the pressing wire pipe space of the stent and the locked coil pipe space (54′) of the multi-porous inner pipe (51) at the same time. The far and proximal openings (516d, 516p) can supply the single opening for the stayguy (70) and also supply the common opening for the stayguy (70) and the pressing wire of the stent (98). The side openings (516d, 516c, 516p) can be used as the channel of the stayguy (70) passing in and out the pore spaces (52, 54, 54′) of the inner pipe and as the drainage of the gas and liquid, e.g. the angiography in the operation. One or more side openings might be on the same level. The strengthening ring (55), which is made from the metal or polymer material with good flexibility and high strength, can be located around the side openings (516d, 516c, 516p) of the inner pipe to increase the sliding of the stayguy and to reduce the possibility of the cut between the stayguy and the inner pipe. The metal strengthening ring (55) can be used as the mark of impervious X-ray.

The plaited strengthening net (56) can be located inside the inner pipe, while the side openings (516d, 516c, 516p) of the inner pipe can open at one mesh of the plaited strengthening net (56).

Still referring to FIG. 1a and FIG. 1b, the medium section (514) of the inner pipe is located nearside the distal segment (513) of the inner pipe. The arched medium section (514) of the inner pipe makes up its concave plane (517) and convex plane (518). The arched medium section (514) of the inner pipe composes the reference plane. The medium section (514) of the inner pipe is not connected with the self-expanding stent (1). The inside and outside diameters of this section can be greater than those of the distal segment (513) of the inner pipe, but its outside diameter must be less than that of the compressed self-expanding stent. With its inside diameter increasing, the friction between the stayguy (70) and the locked coils (75) will decrease. With the increasing of the inside and outside diameters, the inner pipe (51) enhances its flexing resistance. Under external force, the natural arched medium section might be deformed.

Still referring to FIG. 1a and FIG. 1b, the close segment (515) of the inner pipe is straight. It is the extension nearside the medium section (514) of the inner pipe. The close segment (515) and the medium section (514) of the inner pipe can be the spring tube or the plaited strengthening net (56). The near-end (511) of the inner pipe is connected with the nearside controller (80).

Referring to FIG. 1a, combining with FIG. 2a, FIG. 2b, FIG. 2c, FIG. 2d, the texturing tube (61) of the delivery device (2) for the self-expanding stent in present invention might be: 1. An independent texturing tube (61), in which thread with 0.035″ outside diameter can pass through, is set up in the monaulic inner pipe (51). The texturing tube (61) is parallel with the axes of the inner pipe all the time. It comes out from the far-end (512) and near-end (511) of the inner pipe, and stands at the convex plane (518) in the medium section (514) of the inner pipe. The far-end of the texturing tube (611) is connected with the pipe pore (652) of the pipe head (65), while the near-end is connected with the branch texturing tube (86) of the near-end controller (80); 2. A small circular texturing tube (53) forming the texturing tube (61) affixed to the inner pipe, in which thread with 0.035″ outside diameter can pass through, is set up in the biforous or multi-porous inner pipe (51). The small circular texturing tube (53) stands at the convex plane (518) in the medium section (514) of the inner pipe. The far-end of the small circular texturing tube (531) is connected with the pipe pore (652) of the pipe head (65), while the near-end (532) is connected with the branch texturing tube (86) of the near-end controller (80).

Referring to FIG. 1c, combining with FIG. 1a, FIG. 1b, the pipe head (65) of the delivery device (2) for the self-expanding stent in present invention is located outside the far-end (512) of the inner pipe. It might be a part of the inner pipe. The pipe head (65) is a hollow cone with streamline shape. Its back-end is the large end (654) which is connected with the far-end (512) of the inner pipe, while the front-end is the small end (653) which has the pipe pore (652) connecting with the texturing tube (61) or the small circular texturing tube (53). The small end (653) is a hollow conical thin pipe with a soft and thin pipe wall (655). The front segment of the pipe head (65) has one or more side openings (657) and is connected with the pore spaces (52, 54, 54′) which are linked to the far and near part of the inner pipe to provide gas exhaust and the locked coil puncture after flushing. The large end (654) is connected with the far-end (512) of the inner pipe. The pipe head (65) is made up of flexible polymer material containing radiopaque material or embedding the mark of impervious X-ray.

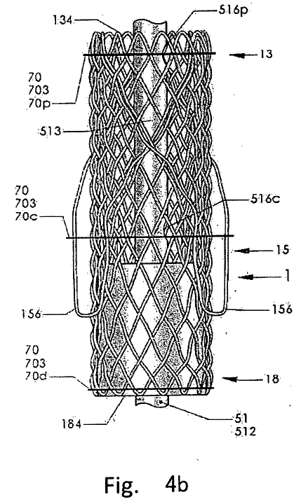

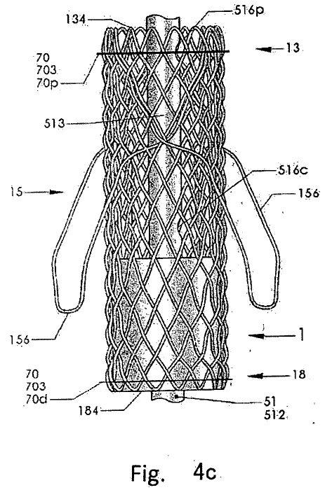

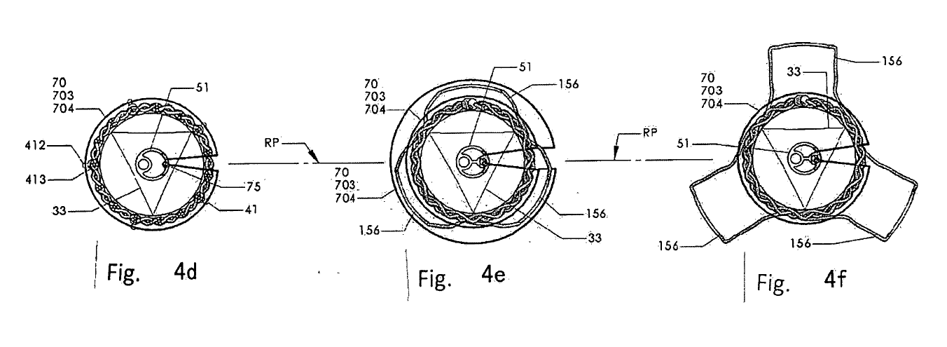

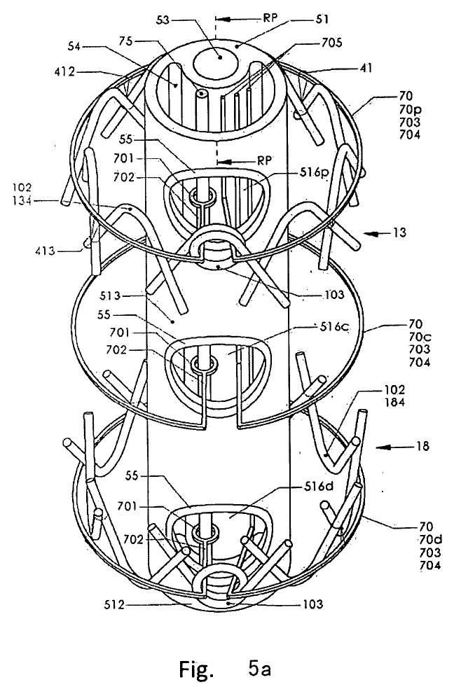

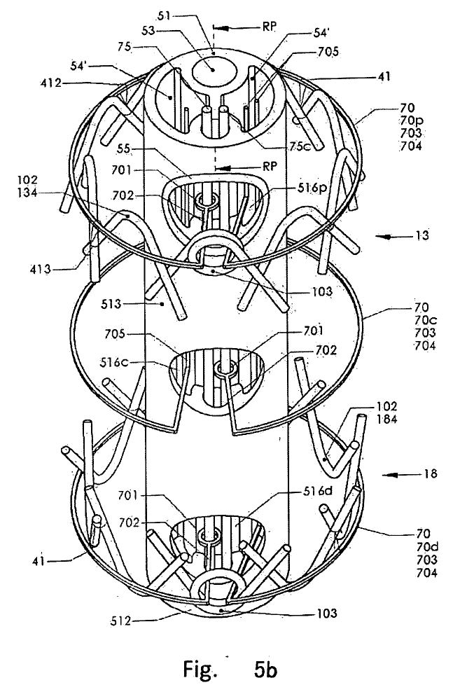

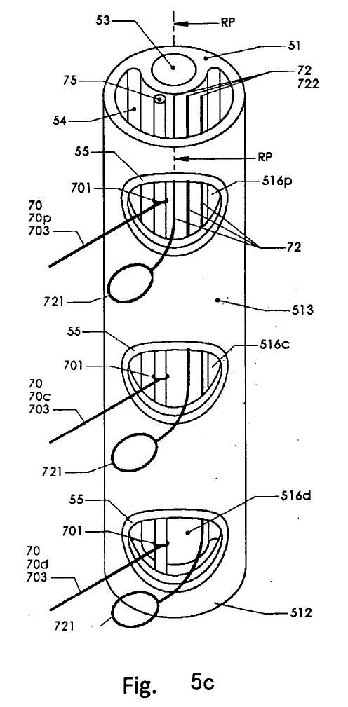

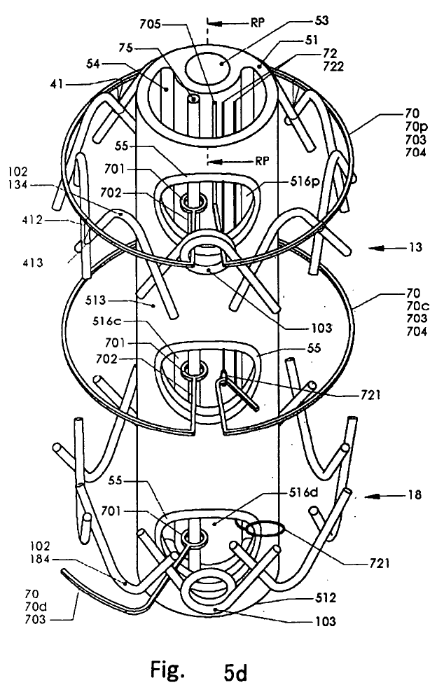

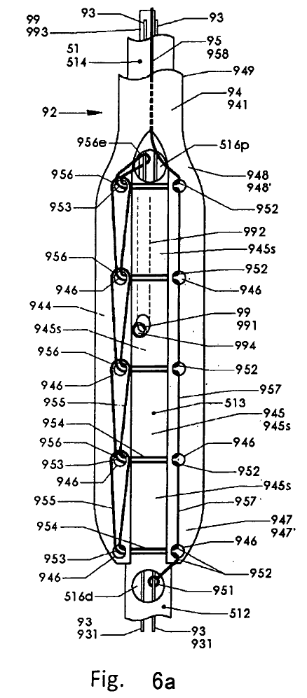

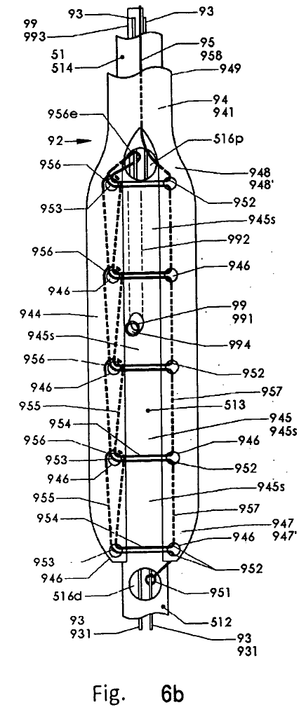

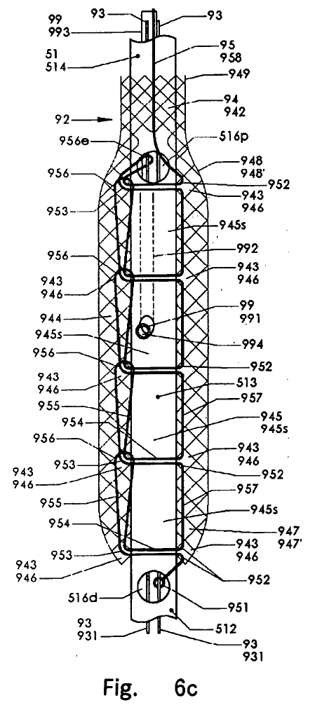

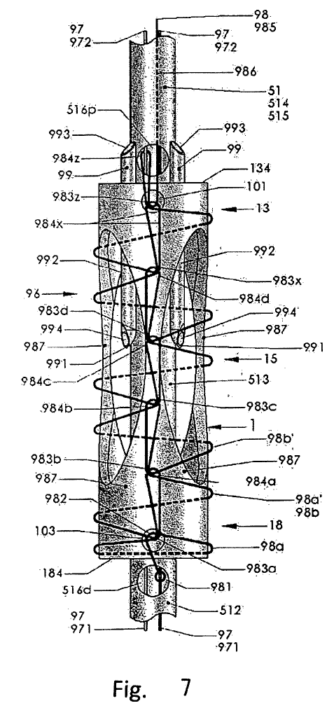

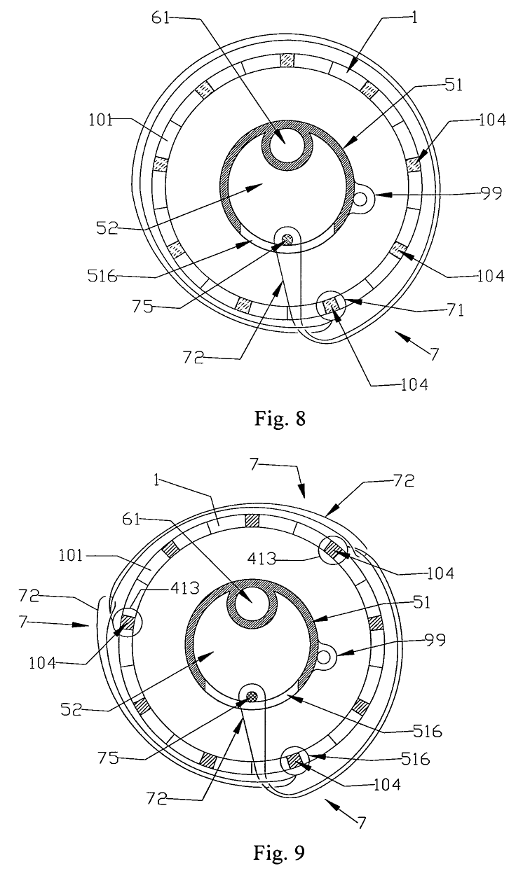

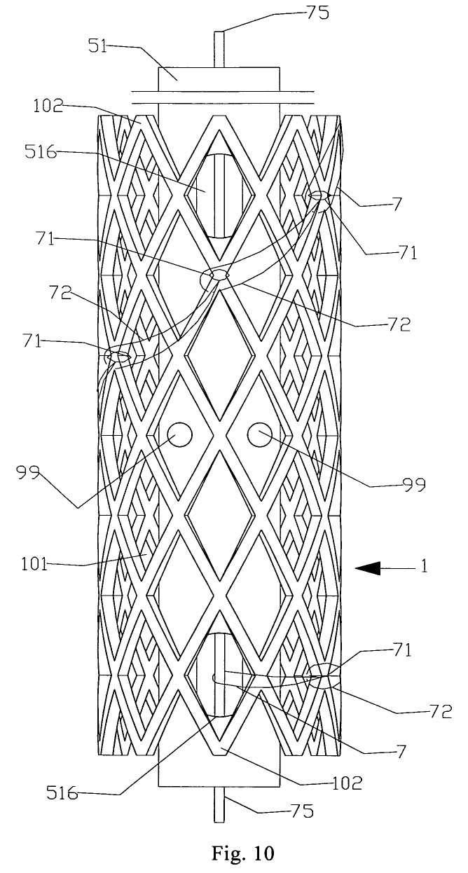

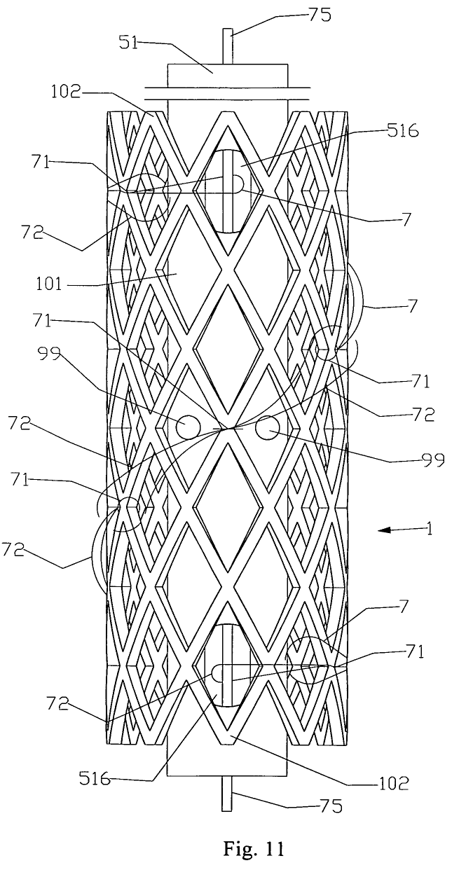

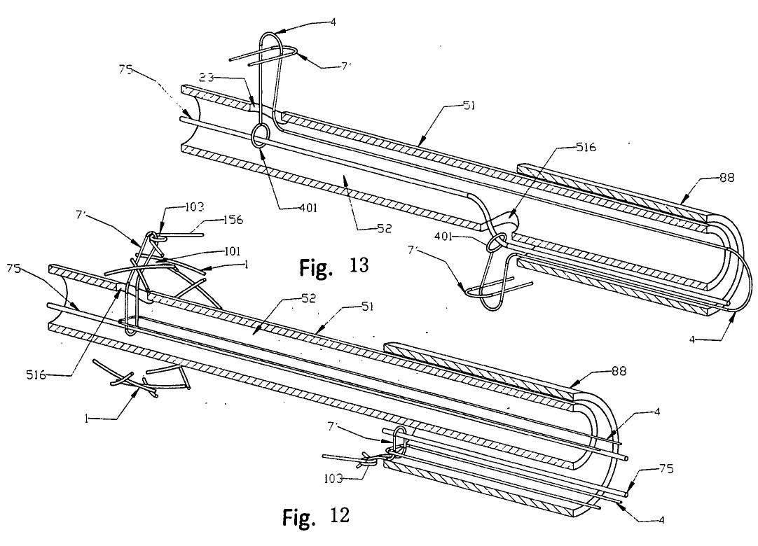

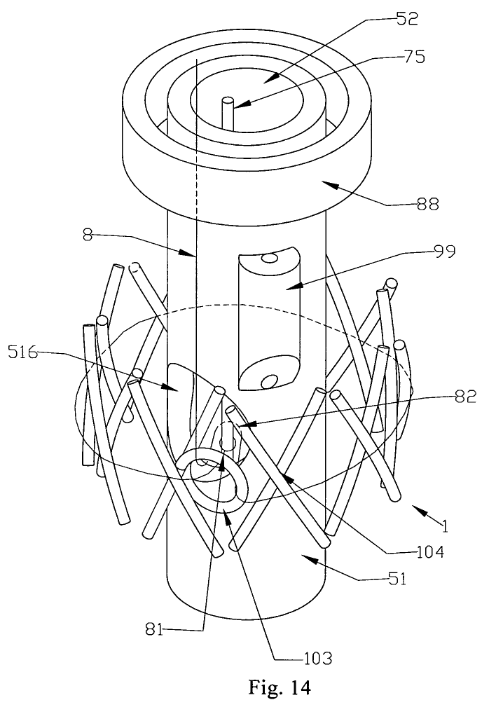

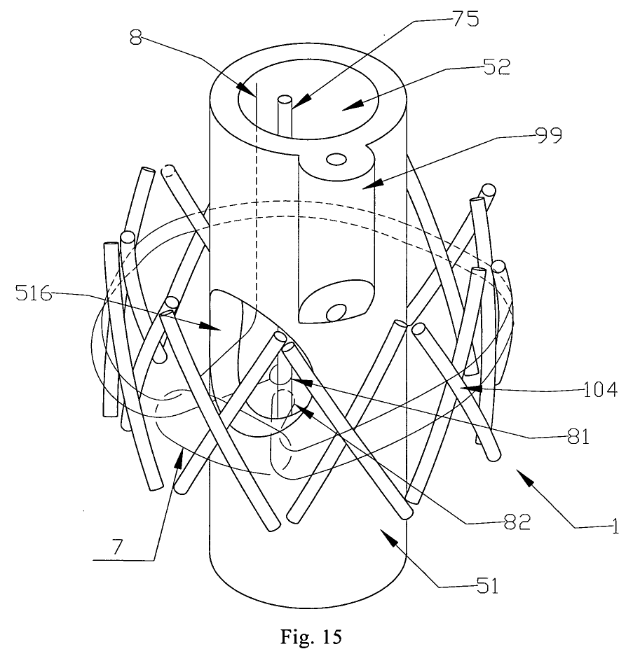

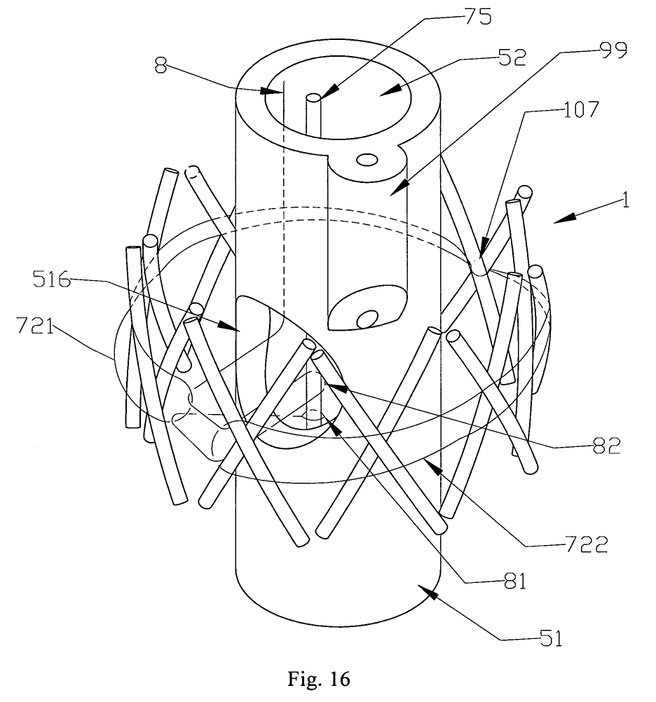

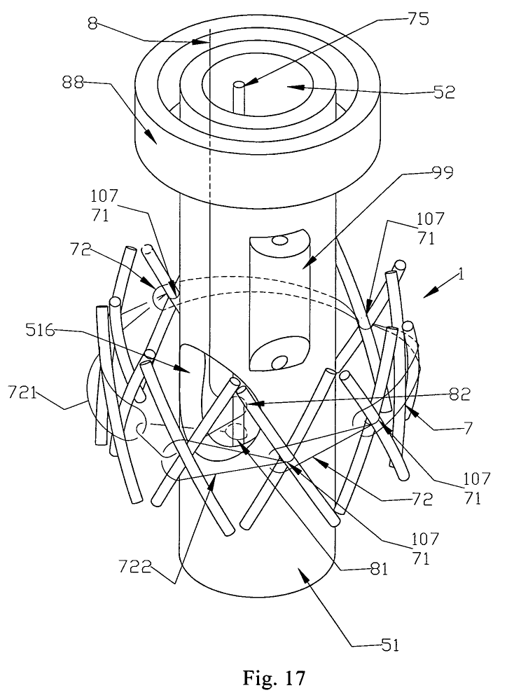

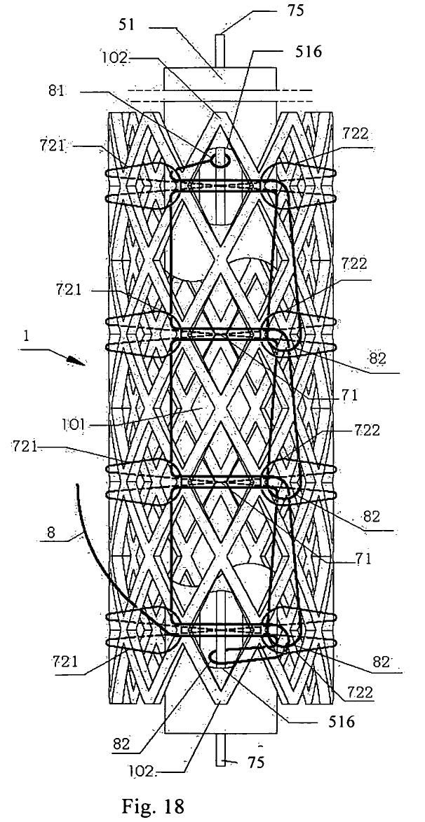

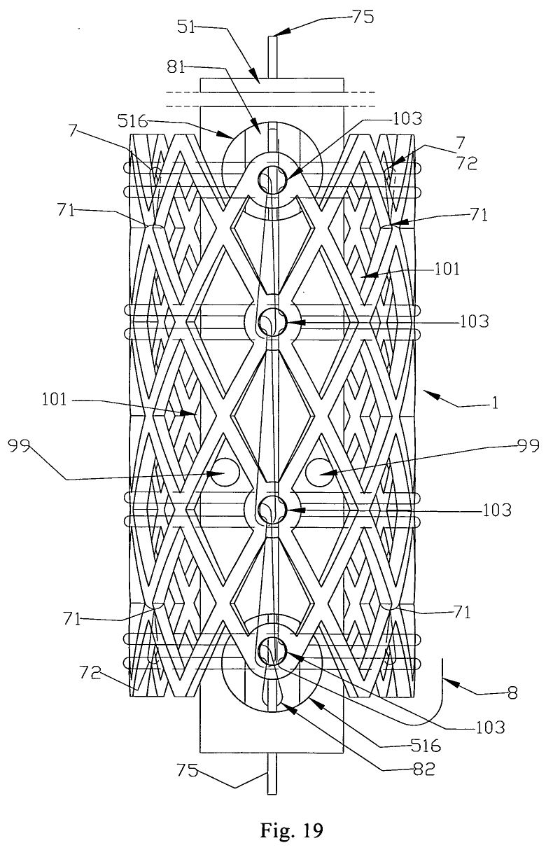

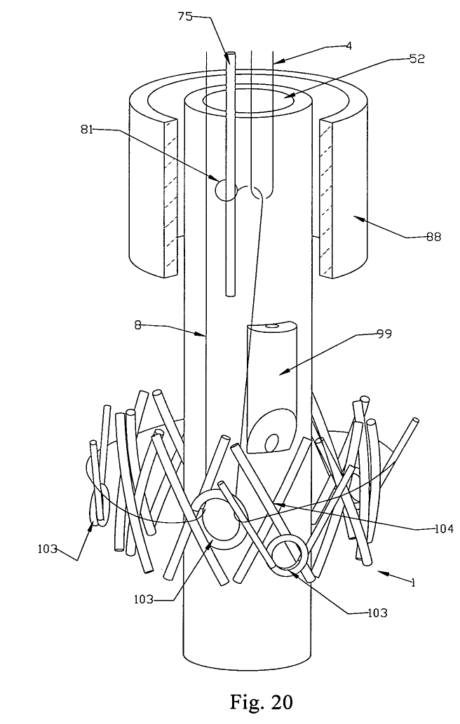

Referring to FIG. 1a, FIG. 1b, FIG. 1d, combining with FIG. 4a, FIG. 4b, FIG. 4c, FIG. 4d, FIG. 5a, FIG. 5b, FIG. 5c, FIG. 5d, the stayguy (70) of the delivery device (2) for the self-expanding stent in present invention is made up of threads of polymer or metal material. Good flexibility, no or little plastic deformation, no or little extension under pulling, no or little cold plastic deformation is required. Every stayguy (70) has a stayguy cycle (701). The stayguy cycle (701) can be a 360° closed thin cycle or an open semi-cycle with 180° folded tail-end of double threads, which is located at the far-end (702) of the stayguy. The stayguy cycle (701) is in one of the side openings (516d, 516c, 516p) of the inner pipe and is locked by the passing locked coil (75). The outside segment (703) is the extension of the stayguy cycle (701), and located outside the side openings (516d, 516c, 516p) of the inner pipe. The outside segment (703) can be double threads or single thread. In order to assemble the self-expanding stent onto the delivery device (2), the outside segment (703) should puncture the opening of the deformable unit (101) or the arched inflexion (102) or the closed line eye (103) of the stent (1) to reach the outside of the stent at least once, and then pass through other deformable unit (101) and the flexible connect collar (41) of the stent. Afterwards, the outside segment (703) puncture the same or different opening of the deformable unit (101) or the arched inflexion (102) or the closed line eye (103) back to the inside of the stent to compose the lasso (704). The distal segment (705) is the extension of the outside segment (703) in the direction of the near-end (511) of the delivery device. The distal segment (705) returns to the same or different above-mentioned far-side opening (516d), intermediolateral opening (516c), proximal opening (516p), and is located inside the distal segment of the inner pipe (513). The distal segment (705) can also be located inside the special stayguy pore spaces (54, 54′) of the distal segment of the inner pipe (513). The medium segment (706) can still be double threads or become a single thread. It also can be connected with other material in certain length for the good flexibility and no extension under the same pulling. The close segment (707) is the extension of the medium segment (706) in the close segment (515) of the inner pipe in the direction of the near-end (511) of the delivery device. The close segment (707) comes out from a relative branch pipe of the stayguy (81d, 81c, 81p) of the near-end controller (80). Every delivery device (2) has one or more stayguys (70, 70d, 70c, 70p). Every stayguy cycle (701) is locked by the same or different locked coils (75, 75c). Every outside segment (703) of stayguys (70d, 70c, 70p) comes out of the far-side opening (516d), the intermediolateral opening (516c), the proximal opening (516p), respectively, and returns back to each opening after it surrounds the stent. Each near-end (708) of the stayguy comes out of each stayguy branch pipe (81d, 81c, 81p) of the near-end controller (80). Each near-end (708) of the stayguy can be put together outside each branch pipe of the stayguy (81d, 81c, 81p) to form one stayguy group (709).

Referring to FIG. 1b, FIG. 1d, combining with FIG. 2a, FIG. 2b, FIG. 2c, FIG. 2d and FIG. 5a, FIG. 5b, FIG. 5c, FIG. 5d, one, two or more locked coils (75, 75c) can be installed in the delivery device (2) for the self-expanding stent in the present invention. Every locked coil can lock one or more stayguys (70, 70d, 70c, 70p). Two or more sets of the locked coils (75, 75c) might work together or work alone. A locked coil (75) is located inside the pore space (52) of the inner pipe or inside the locked coil pore space (54, 54′). Two or more locked coils (75, 75c) can be located inside the same pore space (52, 54) of the inner pipe or each special locked coil pore space (54′). Two or more locked coils (75, 75c) occupy one core space but they have the same length of the far-end. Every far-end (751) of the locked coil (75) exceeds the far-side opening (516d) of the inner pipe. The locked coil (75) punctures the stayguy cycle (701) in the distal segment to avoid its pulling out of the side openings (516d, 516c, 516p) of the inner pipe. The locked coil (75) extends in the direction of nearside and comes out of the branch pipe of the locked coil (83) of the nearside controller (80). One or more locked coil (75) comes out of the same or different branch pipe of the locked coil (83). The near-end (752) of the locked coil is connected with the sliding-jig head (844) of the branch pipe of the locked coil (84). The locked coils (75, 75c) can slide inside the inner pipe (51).

Referring to FIG. 1d, the near-end controller (80) of the delivery device (2) for the self-expanding stent in the present invention is connected with the near-end (511) of the inner pipe. The near-end controller (80) contains the main pipe and many branch pipes (81d, 81c, 81p, 84, 85, 86) linked to the main pipe. These branch pipes are connected with the versatile pore space (52) of the inner pipe (51) or the special pore spaces (54, 54′). These branch pipes might be on the axes of the inner pipe (51) or might be branched off with certain angle. They contain one or more branch pipes of the stayguy (81, 81d, 81c, 81p), one or more branch pipes of the locked coil (84), one branch pipe of flushing and angiography (85) which is connected with above-mentioned two branch pipes (81, 84) and the versatile pore space (52) or each special pore space (54, 54′) of the inner pipe; one or more branch pipes of the thread (86) which are connected with above-mentioned three branch pipes (81, 84, 85) and the versatile pore space (52) or each special pore space (54, 54′) of the inner pipe. The branch pipe of the thread (86) can also be connected with the core space of the texturing tube (61) or with the small circular 0.035″ thread space (53) of the multi-porous inner pipe only, instead of the above-mentioned three branch pipes 81, 84, 85 and the versatile pore spaces (52, 54) of the inner pipe.

The branch pipe of the stayguy (81) can be one, two or more. The branch pipe of the stayguy (81) can be located beside or on the axes terminal of the near-end controller (80). Generally, every branch pipe of the stayguy (81) is passed by one stayguy. The far-end stayguy (70d) comes out of the far-end branch pipe of the stayguy (81d), the medium section stayguy (70c) comes out of the medium section branch pipe of the stayguy (81c), and the near-end stayguy (70p) comes out of the near-end branch pipe of the stayguy (81p). Two or more stayguys (70) can also come out of the same branch pipe of the stayguy (81). The membrane (811) which is made up of flexible polymer materials and exists inside the branch pipe of the stayguy (81) can prevent the blood backstream and has a pore (812) as the passage of the stayguy (70) in the middle. The branch pipe of the stayguy (81) has a stayguy tightner (82) to fasten the stayguy (70) on the branch pipe of the stayguy (81) at some special position. The relationship between the branch pipe of the stayguy (81) and the stayguy tightner (82) can be the same as that of the male and female threads. The stayguy is tied between the male and female threads. The stayguy tightner (82) can be a solid plug, which stuff inside the opening of the stayguy and lock the stayguy at the opening. The stayguy tightner (82) can also be a revolving plug (822) with a stayguy passage (821). When the revolving plug (822) and the branch pipe of the stayguy (81) rotate, the stayguy is locked at the branch pipe of the stayguy.

One or more branch pipes of the locked coil (84) can be on the near-end controller (80). The branch pipes of the locked coil (84) can be on the axes terminal or on the profile of the near-end controller (80). A membrane (831) which is made up of flexible polymer material and has a pore (832) as the passage of the locked coil (75) in the middle is set up on the connector (83) between the main pipe and branch pipe of the locked coil (84) to prevent the blood backstream. The connector (83) on the main pipe has a binding mechanism (833), such as internal threads, which can be connected with the binding mechanism (842) of the branch pipes of the locked coil (84), such as outside threads. Under unfixed conditions, the locked coil (75) can slide between the branch pipes of the locked coil (84) and the inner pipe (51). A sliding-jig (843) can be installed inside the branch pipes of the locked coil (84) and can slide inside the core space (841) of the branch pipes of the locked coil (84). The front-end (844) of the sliding-jig is connected with the near-end (752) of the locked coil. The back-end of the sliding-jig stretches out of the pipe and forms the operating grip. A slot is set up in the middle of the sliding-jig. Far-end locating pin-pole and near-end locating pin-pole are set up on the branch pipe of the locked coil corresponding to the above slot. Two locating pins (848) can pass through the two locating pin-poles on the branch pipe of the locked coil and the slot on the sliding-jig and connect the branch pipe of the locked coil with the sliding-jig. Through fixing or relieving the locating pins, we can determine whether the sliding-jig and the linked locked coil slide or not, and control the distance of their sliding. The locating pin (848) can be classified as the far-end locating pin (848d) and the near-end locating pin (848p). The distance between the two locating pins is less than that of the corresponding openings of the inner pipe. One or more locating pins (848) can prevent the movement of the locked coil (75) toward nearside. The locked coil (75) can move a certain distance toward nearside when one of the locating pins (848) is removed.

The branch pipe of flushing and angiography (85) is set up on the near-end controller (80), and is allocated with one switch (851).

One or more branch pipes of the thread (86) can be installed on the near-end controller (80). The branch pipe of the thread (86) is set up on the back-end of the axes of the near-end controller (80). The branch pipe of the thread (86) is separated from the above three branch pipes (81, 84, 85) and the versatile pore space (52) of the inner pipe, which is connected only with the texturing tube (61) or the pipe space of the texturing tube (53). Made up of polymer materials, a seal membrane can be installed on the branch pipe of the thread (86). The seal membrane has a small pore through which the thread can pass after deformation. Under normal conditions, the pore is closed to leaks no blood.

Referring to FIG. 1c, the B-mode ultrasonic probe (87) is set up on the back-end (654) of the pipe head (65) or on the distal segment (513) of the inner pipe in the delivery device (2) for the self-expanding stent in present invention. For example, one or more B-mode ultrasonic probes (87) can be set up selectively near the far lateral opening (516d) or proximal opening (516p). The thread (871) of the B-mode ultrasonic probe (87) adheres to the inner pipe (51) and passes to the near-end controller (80) with the joint (872).

Referring to FIG. 6a, FIG. 6b, FIG. 6c, FIG. 7, the lateral texturing tube (99) is also set up in the delivery device for the self-expanding stent in present invention. The lateral texturing tube (99) is set up coordinating with the external protection apparatus. One or more lateral texturing tubes (99), through which threads with outside diameter of 0.014″ can pass, can be fastened selectively outside the distal segment (513) of the inner pipe. The lateral texturing tube (99) begins from the outside of the medium section of the distal segment (513) of the inner pipe and extends to the medium segment (514) of the inner pipe at least along the proximal direction of the inner pipe (511) and the close segment (515) of the inner pipe or near-end controller (80). The lateral texturing tube (99) can be connected with the medium segment (514) and the close segment (515) of the inner pipe, while it can also not be connected with them. The far-end (991) of the lateral texturing tube (99) is installed in the distal segment (513) of the inner pipe, between the far lateral opening (516d) and the proximal opening (516p) of the inner pipe, and is not fastened to the inner pipe in a free active state, with the length of several millimeters to apart from the distal segment (513) of the inner pipe. The medium section (992) of the lateral texturing tube (99) is fastened to the distal segment (513) of the inner pipe, and the near-end (993) of the lateral texturing tube (99) is installed nearside the proximal opening (516p) of the inner pipe. The size of the pipe pore (994) of the lateral texturing tube (99) should ensure the thread with diameter of 0.014″ to pass through at least. The angle of rotation between the lateral texturing tube (99) and the far lateral opening (516d), the proximal opening (516p) of the inner pipe can be determined in advance. The angle of rotation between the two or more lateral texturing tubes (99) can also be determined in advance. The far-ends (991) of different lateral texturing tubes (99) can be installed at the same level or not. For example, a left lateral texturing tube (99) angulates about 60° (45-75°) with the lateral openings (516d, 516p) of the inner pipe on the section. Another right lateral texturing tube (99) angulates about 180° with the lateral openings (516d, 516p) of the inner pipe. The bilateral texturing tubes (99) angulate about 120° with each other. Either bilateral texturing tubes (99) can exist without the other, for example, the right lateral texturing tube (99) angulates about 120-180° with the lateral openings (516d, 516p) of the inner pipe, or the left lateral texturing tube (99) angulates about 0-60° with the lateral openings (516d, 516p) of the inner pipe. The strength is enhanced for the 0.014″ lateral texturing tube (99) near the proximal opening of the inner pipe. The position and length of the 0.014″ lateral texturing tube (99) ensure the usage of shorter (1.5 m) thread with the diameter of 0.014″ to exchange quickly.

Referring to FIG. 3a, combining with FIG. 1a, FIG. 1b, the medium pipe (88) is set up in the delivery device (2) for the self-expanding stent in present invention. The medium pipe (88) is an independent tubular structural part and located outside the inner pipe (51) along which it can slide. The inside diameter of the medium pipe (88) is slightly greater than the outside diameter of the inner pipe (51) and less than or equal to the outside diameter of the compressed self-expanding stent. The far-end (881) of the medium pipe doesn't reach the proximal opening (516p) of the inner pipe, while the near-end (882) of the medium pipe connects with the near-end controller (80). A stayguy (89) of the medium pipe is set up inside the medium pipe (88). The far-end (891) of the stayguy (89) of the medium pipe is fastened to the far-end (881) of the medium pipe to form the fixed point (893). The near-end (892) is drawn out of the lateral opening (884) of the near-end (882) of the medium pipe. The far-end fixed point (893) of the stayguy (89) of the medium pipe is set up on the same plane or side as the lateral opening (884) of the near-end (882) of the medium pipe. When we pull the stayguy (89) of the medium pipe, the strain is increased and the medium pipe (88) is bent. And then the stayguy (70) slides naturally to the curving concave plane (517) which is favorable for the passage of the delivery device through aortic arch. Made of silica gel, the shrink-ring which can slide along the inner pipe when loosened and can be fixed on certain position of the inner pipe when tightened is installed inside the near-end (882) of the medium pipe and the lateral opening (884). The transparent or semitransparent medium pipe (88) facilitates the removing of air bubble before operation. The medium pipe (88) can also be used to enhance the strength of the close segment (514, 515) of the inner pipe. The medium pipe (88) is a cone-shaped conduit whose distal segment is thinner and close segment is thicker. Therefore, the delivery device acquires the motivation of the close segment and the bending property of the medium, distal segment at the same time. The medium pipe (88) is a plaited strengthening net. The medium pipe (88) slides along the inner pipe and the far-end (881) of it can push the compressed stent (1) out of the outer sheath (90).

Referring to FIG. 3b, FIG. 6a, FIG. 6b, FIG. 6c, FIG. 7-FIG. 20, the external protection apparatus of the delivery device for the self-expanding stent in present invention can be chosen from the following schemes:

1. Outer Sheath (90)

Referring to FIG. 3b, combining with FIG. 1a, FIG. 1b, made of polymer materials, the outer sheath (90) owns a tubular structure. The transparent or semitransparent outer sheath (90) facilitates the removing of air bubble before operation. The pipe wall of the distal segment (901) of the outer sheath is thin. The inside diameter of the outer sheath is slightly greater than the outside diameter of the compressed self-expanding stent (1), while its length is slightly greater than that of the compressed self-expanding stent (1). The distal segment (901) of the outer sheath is compressed to protect the stent (1). The far-end (902) of the outer sheath has the mark of impervious X-ray (903) embedded in the pipe wall. The medium segment (904) of the outer sheath can be the same tube as the distal segment (901), or can be a tube with thicker pipe wall and less outside and inside diameter than the distal segment (901). The inside diameter of the medium segment is greater than the outside diameter of the medium pipe, and its length is different from that of the distal segment. Under this condition, between the medium segment and the distal segment, there is a boundary pipe (905) of the medium and distal segments. The proximal segment (906) of the outer sheath is a tube with increscent outside diameter. The inside diameter of the proximal segment is greater than or equal to that of the medium pipe (88) and the opening of flushing/angiography and valve (907) are set up in it. A flexible membrane or pipe (909) is set up in the near-end (908) of the outer sheath and a pinhole (910) is installed in the middle of the flexible membrane. Under normal conditions, the pinhole is closed or only has a tiny diameter. The diameter of the medium pipe (88) can expand after passing through the pinhole (910) to ensure the slide of the medium pipe (88) and the flexible membrane (909) blood-tight.

2. Tearable External Protection Apparatus (92)

Referring to FIG. 6a, FIG. 6b, FIG. 6c, the tearable external protection apparatus (92) contains the locked coil (93) of stayguy, the tearable sheath (94) and the take-up wire (95) of sheath. One inner pipe (51) is set up in the middle of the tearable external protection apparatus (92). The far lateral opening (516d) and the proximal opening (516p) are installed in the distal segment (513) of the inner pipe. The two openings (516d, 516p) can provide an independent opening for the take-up wire (95) of sheath, and also can be a uniform opening shared by the stayguy (70). The medium segment and close segment of the inner pipe possess the same installation as above.

At least one locked coil (93) of the stayguy can slide along the inner pipe (51). The locked coil (93) of the stayguy has a far-end (931) and a near-end (932). The near-end (932) can come out of the near-end controller (80).

The tearable sheath (94) is installed outside the inner pipe (51). The tearable sheath (94) has a structure of circular tube which is concentric with the inner pipe (51). The tearable sheath is located outside the inner pipe (51), the self-expanding stent (1) and the medium pipe (88). The tearable sheath (94) and the inner pipe (51) can slide with each other. The tearable sheath (94) is composed of tubular tube layout (941) or tubular mesh duet (942) knitted by natural or synthetic threads. The pipe wall of the tubular tube layout (941) is sealed. The pipe wall of the tubular mesh duet (942) has meshes (943). The meshed pipe wall can be knitted gauze or crocheted textile. The mesh diameter is less than 1 mm. The mesh (943) is deformable but the length of the same side can not be changed. The distal segment (944) of the tearable sheath (94) is at the same level of the distal segment (513) of the inner pipe, and has an axial or longitudinal opening (945). Bilateral sheath of the longitudinal opening (945) has a take-up opening (946), which is an opening of the sealed pipe wall or an eye of the meshed pipe wall (943). The surroundings the mesh (943) of the opening in both sides of the mesh duet is sealed and integral. The diameter of the distal segment (944) can be equivalent to the circumference of the distal segment. The diameter and circumference of the far-end (947) and the near-end (948) of the distal segment (944) can be different. They constitute the conical far-end (947′) and the conical near-end (948′) under curling, respectively. The far-end (947) of the tearable sheath (94) has a shorter diameter and forms warhead streamline type. The longitudinal opening (945) passes through the far-end (947). The medium segment (949) of the tearable sheath (94) is an integral pipe or pipe net, whose diameter and circumference are less than or equal to that of the distal segment (944). The stent (1) can be clipped between the distal segment (513) of the inner pipe and the distal segment (944) of the tearable sheath. The stent (1) is not displayed in FIG. 6a, FIG. 6b and FIG. 6c. The pattern of the combination of the stent (1) and the inner pipe (51) is the same as that of the above.

A longitudinal opening (945) is installed in the distal segment (944) of the tearable sheath and a take-up opening (946) is nearside the longitudinal opening. With the assistance of the take-up wire (95) of the sheath, the longitudinal opening (945) of the distal segment (944) of the tearable sheath can be hold down temporarily and the stent (1) inside is compressed longitudinally. The far-side and nearside of the longitudinal opening (945) of the distal segment (944) of the tearable sheath are connected with the inner pipe (51) temporarily. Now, the tearable sheath (94) and the inner pipe (51) can not slide with each other. The stent (1) is limited between them longitudinally. A far-end take-up collar (951) of the take-up wire (95) of sheath passes through one of the far lateral opening (516d) or the proximal opening (516p) of the inner pipe and is locked by the passing locked coil (93) of the take-up wire. The take-up wire (95) of sheath passes through the stent (1) or the outside stent (far-side or nearside). The take-up wire (95) of sheath couples and passes through the take-up opening (946) on both sides of the longitudinal opening (945) of the distal segment (944) of the tearable sheath to form a single line inflexion (952) in opposite direction on the first side and a double line inflexion (953) on the second side at the same time. The double line vertical segment (954) is located vertically at the longitudinal axis of the sheath between the single line inflexion (952) and double line inflexion (953). The couple take-up wire (95) of sheath constitutes double parallel segment (955) along the longitudinal axis from the double line inflexion (953) to the adjacent take-up opening (946), forming a take-up semi-cycle (956). The in-process is shown in FIG. 6a, FIG. 6b and FIG. 6c. The other head of the take-up wire (95) of sheath goes from the single line inflexion (952) to far-side or nearside to constitute one single parallel segment (957) and couples at the next take-up wire opening (946) and then passes through the take-up wire (946) on both sides of the longitudinal opening (945) and the above take-up semi-cycle (956) to another adjacent take-up wire opening (946) to form another take-up semi-cycle (956). The take-up semi-cycle (956) is locked by the subsequent double line inflexion (953) of the take-up wire (95). Repeating as follows: the single line inflexion (952)—the double line vertical segment (954)—the double line inflexion (953)—double parallel segment (955)—the take-up semi-cycle (956)—the single parallel segment (957). The length of the double line vertical segment (954) represents the degree of the tightness of the take-up wire (95) or the level of the radial compression of the stent (1). The last take-up semi-cycle (956e) enters another lateral opening (516p, 516d) of the inner pipe and is locked by the same or another locked coil (93) of the take-up wire. This take-up semi-cycle (956e) can pass through the stent (1) or is located at the nearside of the external stent if it does not pass through it. The take-up wire (95) of sheath is a soft thin thread. Every double line inflexion (953) and every take-up semi-cycle (956) can deform or stretch. The position of each segment of the take-up wire (95) of sheath can be changeable. The proximal segment (958) of the take-up wire of the sheath can pass the inside or outside of the proximal segment (949) of the tearable sheath and lead to the nearside (511) of the releasing mechanism. The near-end (959) of the take-up wire of the sheath is fixed on the near-end controller (80) temporarily. Between the two sides of the longitudinal opening (945) of the distal segment (944) of the tearable sheath, and between the two adjacent double line vertical segments (954) of the take-up wire (95), a small temporary opening (945s) of sheath is installed to connect out and in. The longitudinal opening (945) can be divided into several small temporary openings (945s). When the longitudinal opening (945) of the tearable sheath (94) is hold down, one or more lateral texturing tubes (99) or threads, e.g. 0.014″ texturing tube, can pass from the inner pipe (51), inside to outside, through the uncovered part of the stent (1), through the small temporay opening (945s) or the longitudinal opening (945), and enter the opening of the lateral branch of artery, e.g. the opening of coronary artery. The lateral texturing tube (99) or threads on the same section can only come out in one direction when there is one longitudinal opening (945).

The working principle of the tearable external protection apparatus (92) is as follows:

a. If the near-end (932) of the locked coil of the take-up wire is drawn outwards, the locked coil (93) of the take-up wire slides to the near-end. The far-end (931) of the locked coil of the take-up wire slides out of the far-end take-up collar (951) or the last take-up semi-cycle (956e). The take-up wire (95) of the sheath isn't locked by the passing locked coil (93) of the take-up wire and is released.

b. If we draw the near-end (959) of take-up wire, the double line (953, 954, 955 and 956) can retreat from the double line semi-cycle (956) which is released after being locked. And then the linkage of the both sides of the longitudinal opening (945) of the tearable sheath (94) is released in opposite order. The tearable sheath (94) becomes the real longitudinal opening (945), and the take-up wire (95) of the sheath can be all recovered to the near-end of the delivery device.

3. Sintered and Unhitched Hold-Down Mechanism (96)

Referring to FIG. 7, the sintered and unhitched hold-down mechanism (96) contains the locked coil (97), the pressing wire (98) of the stent. The structure of the inner pipe (51) in the sintered and unhitched hold-down mechanism (96) is identical with the above. The monaulic inner pipe (51) has a large circular versatile pore space (52); the biforous inner pipe (51) has two pipe spaces, a texturing tube 0.035″ (53) providing the passage of thread 0.035″ and a large semi-lunar versatile pipe space (54) providing the passage of the locked coil (97) and pressing wire (98) of the stent. The multi-porous inner pipe (51) has several pipe spaces, a texturing tube 0.035″ (53) providing the passage of thread 0.035″ and several pipe spaces (54′) providing the passage of the locked coil (97) and pressing wire (98) of the stent in their own pipe spaces. At least two openings are set up in the distal segment (513) of the inner pipe, the far lateral opening (516d) and the proximal opening (516p). The two openings connect with the inner pipe space (52) of the monaulic inner pipe, or connect with the versatile pipe space (54) of the biforous inner pipe, or connect with the pipe spaces (54′) of the pressing wire of the stent and the pipe spaces (54′) of the locked coil of the multi-porous inner pipe at the same time. The distance between the two openings is probably equal to the length of the compressed stent. The two lateral openings (516d, 516p) serve as the single opening for the pressing wire of the stent (98) and also as the common opening for the pressing wire of the stent (98) and the stayguy (70).

Inside the pipe space (52) of the monaulic inner pipe, or the pipe space (54) of the biforous inner pipe, or the pipe space (54′) of the multiporous inner pipe, one or more locked coils (97) can pass through from the far-end (512) to the near-end (511). The locked coil (97) has a far-end (971) and a near-end (972). The near-end (972) comes out of the branch pipe of the near-end controller (80). The locked coil (97) can slide along the inner pipe space (52) or common pipe space (54) or pipe space of the locked coil (54′). At the same level or section, two lateral texturing tubes (99) can export the thread 0.014″ from the medium section of the stent (1) and the segments of the pressing wire (98) of the stent.

One concentric stent (1) to be implanted is installed outside the distal segment (513) of the inner pipe. The stent (1) isn't included in the delivery device (2), but is located in the hold-down mechanism (96) before it is released. The stent (1) can be fixed temporarily by the above stayguy (70) on the same lateral opening or other lateral openings (516d, 516c, 516p) of the inner pipe (51).

The stent (1) can be compressed radially by the pressing wire (98) of the stent temporarily. The pressing wire (98) of the stent is a soft deformable thin thread and is located outside the stent (1). One end of the pressing wire (98) of the stent has a far-end line collar (981), which passes one of the far lateral openings (516d) or the proximal opening (516p). The far-end line collar enters the inner pipe space (52) or common pipe space (54) or pipe space of the locked coil (54′) and is locked by the passing locked coil (97). Before it enters the far lateral opening (516d) or the proximal opening (516p), the far-end line collar (981) passes outside the stent (1) selectively, or the uncovered part of the stent (1). The pressing wire (98) of the stent expands on the outer surface of the stent (1) to forms a single line inflexion (982). The pressing wire (98) of the stent turns into double lines (98a, 98a′), which continue to wrap the stent (1) about 360°. The double lines (98a, 98a′) pass through the above single line inflexion (982) to form the first double line inflexion (983a) and continue to form the first semi-cycle (984a) of the pressing wire. The next segment of the pressing wire (98) of the stent keeps on being the double lines (98b, 98b′), which wrap the stent (1) about 180° in the direction opposite to the above. The double lines (98b, 98b′) pass through the above first semi-cycle (984a) of the pressing wire to form the second double line inflexion (983b) and continue to form the second semi-cycle (984b) of the pressing wire. Repeated as above, the double pressing wires (98) of the stent wrap round the outer surface of the stent (1) once in one direction, and wrap in opposite direction to form the other two double line inflexions (983c, 983d) and the other two semi-cycles (984c, 984d) of the pressing wire. The later double line inflexion (983d) passes through the former semi-cycle (984c) of the pressing wire. Then, a double line unit can repeat countless times. The pressing wire (98) of the stent is a soft thin thread. Every double line inflexion (983) and every semi-cycle (984) of the pressing wire can deform or stretch and their positions can be exchanged. The pressing wire (98) can become double lines in the process of shuttling between any double line inflexion (983a, 983b, 983c, 983d . . . 983x) and any semi-cycle (984a, 984b, 984c, 984d . . . 984x) of the pressing wire. And then the double lines pass through the uncovered part of the stent (1) at the same time, from outside to inside and from inside to outside, and fasten the pressing wire (98) to the stent (1) temporarily. After the last semi-cycle (984z) of the pressing wire passes through the former semi-cycle (984x) of the pressing wire to form the last double line inflexion (983z), it passes through the lateral openings (516d, 516p) of the inner pipe on the other end. Then the last semi-cycle enters the inner pipe space (52) or common pipe space (54) or pipe space of the locked coil (54′) and is locked by the same or another locked coil (97). Before it enteres the lateral openings (516d, 516p) of the inner pipe, the last semi-cycle (984z) of the pressing wire passes outside the stent (1) selectively, or the uncovered part of the stent (1). And then, after the far-end line collar (981) and the last semi-cycle (984z) of the pressing wire are locked by the same or different locked coil (97), drawing the near-end (985) of the pressing wire of the stent can hold down the pressing wire (98) and compress the nether stent (1) radially. When the far-end line collar (981) and the last semi-cycle (984z) of the pressing wire are locked by the same or different locked coil (97), the pressing wire (98) of the stent can wrap the stent (1) for the second time in the same manner selectively. The pressing wire (98) of the stent, which wraps the stent (1) for the second time, is the extension of the same pressing wire. The segment of the pressing wire (98) of the stent which wraps the stent (1) for the second time is installed outside that for the first time. The direction of the pressing wire (98) of the stent which wraps the stent (1) for the second time is opposite to that of the first time and retreats near the beginning of the far-end line collar (981). The last semi-cycle (984z) of the pressing wire of the stent which wraps the stent (1) for the second time can enter the same or different lateral opening (516p) of the far-end line collar (981) and be locked by another locked coil (97). The close segment (986) of the pressing wire of the stent enters selectively between the inner pipe (51) and the medium pipe (88) or enters inside the space of the inner pipe (51). The near-end (985) of the pressing wire connects with the near-end controller (80) temporarily. A concentric medium pipe (88) is installed outside the close segment (514, 515) of the inner pipe. The inside diameter of the medium pipe (88) is greater than the outside diameter of the inner pipe (51), making the inner pipe (51) slide along the inner pipe (51). The pressing wire (98) of the stent passes between the inner pipe (51) and the medium pipe (88).

The working principle of the sintered and unhitched hold-down mechanism (96) is as follows:

a. The pressing wire (98) of the stent is tightened and locked by the locked coil (97). The pressing wire compresses the nether self-expanding stent (1) radially to reduce its diameter, indicating the condition of compression or implantation;

b. Under the above compressed conditions, one or more far-ends (991) of the lateral texturing tubes or lateral threads can pass through the uncovered and deformable unit (101) of the medium or low section (15, 13) of the stent (1) from inside to outside and then reach the outside of the stent (1) between the pressing wires (98) of the stent and enter the entry of the blood vessel branch, e.g. the opening of the coronary artery. After one or more lateral threads enter the blood vessel branch, the location of the rotation direction of the delivery device (2) and the stent (1) can be determined. As the pressing wire (98) occupies little area, the far-end (991) of the lateral texturing tube can draw several different lateral threads at different sections and different rotating angles out. The far-end (991) of the lateral texturing tube enters the open area (987) which is located at the segments of the pressing wires (98) of the stent. Even though the sintering of the pressing wires (98) of the stent is unhitched, the lateral threads and the pressing wires (98) of the stent are not locked each other when the pressing wires (98) of the stent moves towards the near-end.

c. One or more locked coils (97) move towards the near-end in turn. The far-end (971) of the locked coil slides out of the last semi-cycle (984z) of the pressing wire of the stent and the far-end line collar (981). Then the pressing wire (98) of the stent is released. Drawing the near-end (985) of the pressing wire towards the near-end (511) releases the sintering of the pressing wires between the double line inflexions (983a, 983b, 983c, 983d . . . 983x) and the semi-cycles (984a, 984b, 984c, 984d . . . 984x) of the pressing wire outside the stent (1) and move all the pressing wires (98) of the stent to the near-end (511). The lateral thread is located at the open area (987) between the pressing wires (98) of the stent and isn't influenced by the movement of the pressing wires (98) of the stent.

4. Hold-Down Mechanism (96) of the Flexible Connect Collar

Referring to FIG. 8, combining with FIG. 9 to FIG. 13, the hold-down mechanism (96) of the flexible connect collar in the present invention contains the inner pipe (51) and the locked coil (75) applied to constitute the implantation system, and at least one flexible connect collar (7) applied to compress the stent. The flexible connect collar (7) is connected with the stent. At least one of the flexible connect-collars passes through the stent and the lateral opening (516) of the inner pipe and is locked by the passing locked coil (75) temporarily.

The flexible connect collar (7) in the present invention is one part of the stent (1) and is knitted by the soft and deformable strings, e.g. DACRON fiber, POLYETHYLENE fiber, PA fiber, POLYPROPYLENE fiber and so on. It has two structures. One is shown in FIG. 8, FIG. 9, FIG. 10, FIG. 11, including the fixed point (71) and free point (72). The fixed point (71) wrap and tie to the arched inflexion of the stent (1) or the closed line eye (103) or the cross-point of the mesh (104) of the stent. The free point (72) expands inside or outside of the stent and constitutes single or double free cycles. Another is shown in FIG. 12, FIG. 13, and is a single closed line collar (7′). Passing through the arched inflexion (102) or the closed line eye (103) or the cross-point of the mesh (104) of the stent, the single closed line collar can slide but not break away.

The first instance of the hold-down mechanism of the flexible connect collar in the present invention is shown in FIG. 8. Containing one single free cycle, the flexible connect collar (7) is applied to press the stent. The free cycle (72) of the flexible connect collar wraps outside the stent (1) under radial compression once. The free cycle passes through a closed eye (103) or the same single free cycle of the stent and a deformable unit (101) of the stent (1) and then passes the lateral opening (516) of the inner pipe and is locked by the passing locked coil (75) of the inner pipe temporarily. If the locked coil (75) slides to the near-end, the free cycle (72) of the flexible connect collar (7) is separated and released from the locked coil (75), while the stent (1) under radial compression reaches the radial extension. Two to three above hold-down mechanisms of the flexible connect collar can be set up at different radial section of the same stent to hold down the whole stent.

The second instance of the hold-down mechanism of the flexible connect collar in the present invention is shown in FIG. 9. The flexible connect collar (7), which is applied to press the stent, contains single free cycle. Each flexible connect collar is distributed circularly outside or inside of the same section of the stent. The adjacent flexible connect collars of the stent under radial compression are connected one by one and wrap around the stent once. At last the free cycle (72) of the flexible connect collar passes through a closed eye (103) or the single free cycle of the first connect collar of the stent and a deformable unit (101) of the stent (1). And then the free cycle passes the lateral opening (516) of the inner pipe (51) and is locked by the passing locked coil (75) of the inner pipe temporarily. If the locked coil (75) slides to the near-end, the free cycle (72) of the last flexible connect collar (7) is separated and released from the locked coil (75), while the stent (1) under radial compression reaches the radial extension. Two to three above hold-down mechanisms of the flexible connect collar can be set up at different radial section of the same stent to hold down the whole stent.

The third instance of the hold-down mechanism of the flexible connect collar in the present invention is shown in FIG. 10. The flexible connect collar (7), which is applied to compress the stent, contains single free cycle. Each flexible connect collar is distributed helically outside the stent. The adjacent flexible connect collars of the stent under radial compression are connected one by one and wrap around the stent at least once. At last the free cycle (72) of the flexible connect collar passes through a closed eye (103) or the single free cycle of the first connect collar of the stent and a deformable unit (101) of the stent (1). And then the free cycle passes the lateral opening (516) of the inner pipe (51) and is locked by the passing locked coil (75) of the inner pipe temporarily. If the locked coil (75) slides to the near-end, the free cycle (72) of the last flexible connect collar (7) is separated and released from the locked coil (75), while the stent (1) under radial compression reaches the radial extension.

The fourth instance of the hold-down mechanism of the flexible connect collar in the present invention is shown in FIG. 11. The flexible connect collar (7), which is applied to compress the stent, contains a flexible connect collar with double free cycle (72) and several flexible connect collars with single free cycle (72). Each flexible connect collar is distributed helically outside the stent and a flexible connect collar with double free cycle is set up in the middle of the stent. The two flexible connect collars of the stent under radial compression expand in opposite direction respectively and connect with the adjacent single free cycle one by one at least once. The last two free cycles (72) of the flexible connect collar expanding in opposite direction pass through a closed eye (103) or certain single free cycle of the above connect collar of the stent and a deformable unit (101) of the stent (1). And then they pass the far lateral or proximal openings (516) of the inner pipe (51) and are locked by the same or different locked coil (75) of the inner pipe temporarily. If the locked coil (75) slides to the near-end, the two free cycles (72) of the last flexible connect collar (7) expanding in opposite direction are separated and released from the locked coil (75), while the stent (1) under radial compression reaches the radial extension.

The hold-down mechanism of the flexible connect collar in the present invention also contains the temporary stayguy (4) and the medium pipe (88) applied to constitute the implantation system. Such a structure is shown in the fifth instance of the hold-down mechanism of the flexible connect collar in the present invention. As shown in FIG. 12, the flexible connect collar applied to hold down the stent is a single closed line collar (7′). One end is fixed to the closed line eye (103) of the stent, another end passes through the stent and the lateral openings (516) of the inner pipe to enter the inner pipe, or is locked by the passing locked coil (75) temporarily when it enters into the inner pipe (51) and the medium pipe (88). The medium section is drawn by the stayguy (4) temporarily. Such hold-down mechanisms of the flexible connect collar can be applied to the two-layer stent with outer tongue. As shown in FIG. 12, the top-end of the outer tongue (156) has a closed line eye (103), which can be connected with at least one flexible connect collar. When the outer tongue (156) is compressed, the flexible connect collar (7′) passes through the inner layer of the stent, or warps around the outer tongue (156). Afterwards, the flexible connect collar passes through the adjacent closed line eye (103) of the outer tongue and the inner layer of the stent (1) and then passes the lateral opening (516) of the inner pipe (51) and is locked by the passing locked coil (75) of the inner pipe temporarily. If the locked coil (75) slides to the near-end, the flexible connect collar (7′) is separated and released from the locked coil (75), while the outer tongue under radial compression reaches the radial extension in prior.

The sixth instance of the hold-down mechanism of the flexible connect collar in the present invention is similar to the fifth instance, as shown in FIG. 13. The difference between them is that the flexible connect collar (7′) (closed line collar) in this instance is fastened by a stayguy instead of being locked by the passing locked coil (75) of the inner pipe. A stayguy collar (401) is set up on the far-end of the stayguy (4) and is locked by the passing locked coil (75) between inner pipe (51) and the medium pipe (88). If the stayguy (4) is loosened, the stent (1) under radial compression reaches the radial extension. If the locked coil (75) slides to the near-end, the flexible connect collar (7′) is separated and released from the locked coil (75).

The hold-down mechanism of the flexible connect collar in the present invention can also be the flexible connect collar (7) which is applied to the hold-down mechanism with double free cycles (no figure). The two free cycles (72) of the flexible connect collars expand in opposite direction respectively and wrap the stent half cycle. The two free cycles pass the same closed line eye (103), the same lateral opening (516) of the inner pipe and are locked by the passing locked coil (75) of the inner pipe temporarily. If the locked coil (75) slides to the near-end, the two free cycles (72) of the flexible connect collar (7) are separated and released from the locked coil (75), while the stent (1) under radial compression reaches the radial extension. Two to three above hold-down mechanisms of the flexible connect collar can be set up at different radial section of the same stent or can be used together with the first instance or second instance.

The working principle of the hold-down mechanism of the flexible connect collar is as follows:

a) Various locking methods of different flexible connect collar are alternative.

a1) The free cycle of the flexible connect collar passes through the lateral opening (516) of the inner pipe and is locked by the passing locked coil (75). (as shown in FIG. 8, FIG. 9, FIG. 10, FIG. 11)

a2) On the basis of (a1), the locked flexible connect collar is not only hitched by the temporary stayguy (4), but also pulled to the near-end. The stayguy (4) is located between inner pipe (51) and the medium pipe (88). The free cycle (72) of the flexible connect collar (7) is locked by the locked coil (75) and is drawn inside or outside the inner pipe by the stayguy (4) at the same time. (as shown in FIG. 12)

a3) Similar to (a2), the free cycle (72) of the flexible connect collar enters the far-end opening (51) of the medium pipe (88) instead of the lateral opening (516) of the inner pipe. (as shown in FIG. 12)

a4) The flexible connect collar is not locked by the inner pipe or medium pipe, but is passed through by the stayguy (4) without being locked. When the flexible connect collar is pulled to the near-end, the line collar (401) on one end of the stayguy (4) is locked by the locked coil (75) of the inner pipe (51) or medium pipe (88). (as shown in FIG. 13)

b) The free cycle (72) of the hitched flexible connect collar (7) compresses the self-expanding stent (1) radially. The whole length of free cycles (72) on every section is 7r times to the diameter of the compressed stent (1).

c) Besides the texturing tube (21) inside the inner pipe (51), lateral texturing tube (99) is inside or on the inner pipe to lead threads to the direction vertical to the inner pipe. The location and rotation of the far-end opening of the lateral texturing tube is a standing relationship with the lateral opening (516) of the inner pipe, hence it has an indirect and adjustable relationship with the stent or self-expanding fenestrated covered stent. The stent has uncovered deformable unit (101) and the fenestrated covered stent has a window through which threads can pass.

d) After the locked coil (75) is loosened, each free cycle (72) of the flexible connect collar (7) is unhitched in turn. Under resilience force, the stent (1) expands and releases quickly.

e) Different from the deformable unit (101), the size and shape of the closed line eye (103) does not change with the diameter of the stent (1), even under the pull of the passing free cycle (72).

5. The Hold-Down Mechanism of the Take-Up Wire

Referring to FIG. 14-FIG. 20, the hold-down mechanism of the take-up wire in present invention can be regarded as a sintered and unhitched hold-down mechanism. It contains the locked coil (75), at least one connect collar on the stent provided generally by the closed line eye (103) and/or at least one flexible connect collar (7), and the take-up wire (8) which is applied to compress the stent. A take-up collar (81) is placed at the far-end of the take-up wire (8) and is locked temporarily by the passing locked coil (75) which is placed inside the inner pipe (51) or between the inner pipe (51) and the medium pipe (88). The take-up wire passes the same or different lateral openings (516) inner pipe at least twice, and wraps among the locked coil (75), the closed line eye (103) or the flexible connect collar (7) and the outside of the stent to form the hold-down mechanism. The near-end of the take-up wire comes out of the near-end of the implantation system and fastens temporarily to the near-end controller of the implantation system.

The first instance of the hold-down mechanism of the take-up wire in present invention is shown in FIG. 14. The hold-down mechanism of the take-up wire has a structure of single take-up wire and single section. The closed line eye (103) is set up on the compressed section of the stent (1) (also can be a closed flexible connect collar (7)). The take-up wire (8) is locked by the locked coil (75) at its far-end take-up collar (81), and passes through a lateral opening (516) of the inner pipe from inside to outside, and the closed line eye (103) (or the closed flexible connect collar (7)) of the stent. And then it wraps outside the stent once, and passes through the same closed line eye (103) (or the closed flexible connect collar (7)) from outside to inside, and the same lateral opening (516) of the inner pipe. It bypasses the locked coil (75) of the inner pipe and forms the take-up semi-cycle (82). Afterwards it comes out of the same lateral opening (516) of the inner pipe from inside to outside, and the same closed line eye (103) (or the closed flexible connect collar (7)) of the stent once more to form a sintered and unhitched hold-down mechanism at certain section of the stent. Passing through the inner layer between the inner-pipe (51) and the medium pipe (88), it comes out of the near-end of the implantation system and is fixed temporarily to the near-end controller of the implantation system.

The second instance of the hold-down mechanism of the take-up wire in present invention is shown in FIG. 15. This hold-down mechanism of the take-up wire also has a structure of single take-up wire and single section. The closed flexible connect collar (7) is set up on the compressed section of the stent (1) (also can be a closed line eye (103)). The take-up wire (8) is locked by the locked coil at its far-end take-up collar (81), and passes through a lateral opening (516) of the inner pipe from inside to outside, and the closed flexible connect collar (7) (or closed line eye (103)) of the stent. And then it wraps outside the stent about one circle, and passes through the same closed flexible connect collar (7) (or closed line eye (103)) from outside to inside, and the same lateral opening of the inner pipe entering the inner pipe. It bypasses the locked coil (75) of the inner pipe and forms the take-up semi-cycle (82). Afterwards it comes out of the same lateral opening (516) of the inner pipe from inside to outside, and the same closed flexible connect collar (7) (or closed line eye (103)) of the stent once more. And then it wraps outside the stent about one circle in opposite direction, and passes through the same closed flexible connect collar (7) (or closed line eye (103)) from outside to inside, and the same lateral opening (516) of the inner pipe (51) entering the inner pipe to form a sintered and unhitched hold-down mechanism at certain section of the stent. Passing through the inner pipe (51), it comes out of the near-end of the implantation system and is fixed temporarily to the near-end controller of the implantation system.

The third instance of the hold-down mechanism of the take-up wire in present invention is shown in FIG. 16. This hold-down mechanism of the take-up wire also has a structure of single take-up wire and single section. A hold-down mechanism of the flexible connect collar which is composed of a flexible connect collar (7) with double free cycles is placed on the compressed section of the stent (1). The fixed-point of the flexible connect collar (7) wraps and ties to a cross-point of mesh wires of the stent. The two free cycles (721, 722) expand along the circumference of the outside surface of the stent to both sides and wraps the stent (1) under radial compression about one circle to form an opposite condition near the propinquity (The two free cycles may not approach to each other and have a far distance, e.g. a half cycle apart or less, but the two free cycles form an opposite condition at the same circumference of the stent.) The take-up wire (8) is locked by the locked coil (75) at its far-end take-up collar (81), and passes through a lateral opening (516) of the inner pipe from inside to outside. Afterwards it passes through the first free cycle (721). Then it curves to the second free cycle (722) and passes through it from outside to inside. The take-up wire passes through the same lateral opening (516) and enters the inner pipe, bypasses the locked coil (75) of the inner pipe and forms the take-up semi-cycle (82). Afterwards it comes out of the same lateral opening (516) of the inner pipe from inside to outside, and the second free cycle (722). Then the take-up wire curves to the first free cycle (721) and passes through it from outside to inside, passes through the same lateral opening (516) and enters the inner pipe to form a sintered and unhitched hold-down mechanism. At last the take-up wire comes out of the near-end of the implantation system passing through the inner pipe (51) and is fixed temporarily to the near-end controller of the implantation system.

The fourth instance of the hold-down mechanism of the take-up wire in present invention is shown in FIG. 17. This hold-down mechanism of the take-up wire also has a structure of single take-up wire and single section. A hold-down mechanism of the flexible connect collar which is composed of several flexible connect collars (7) is placed on a certain compressed section of the stent (1). The fixed points of two flexible connect collars (71) wrap and tie to the same cross-point of mesh wires of the stent. Other fixed points of the flexible connect collars (7) wrap and tie to the cross-point which is placed on the same circumference as the above cross-point. Each free cycle (72) of the flexible connect collar expands along the circumference of the outside surface of the stent to both sides and wraps the stent (1) under radial compression about one circle. Expanding in one direction, the free cycles are connected one by one and the last two free cycles (721, 722) expand in different directions to form an opposite condition near the propinquity (The last two free cycles which expand in one direction may not approach to each other and have a far distance, e.g. a half cycle apart or less, but the two free cycles form an opposite condition at the same circumference of the stent.) The take-up wire (8) is locked by the locked coil at its far-end take-up collar (81), and passes through a lateral opening (516) of the inner pipe from inside to outside. Afterwards it passes the first free cycle (721). Then it curves to the second free cycle (722) and passes through it from outside to inside, passes through the same lateral opening (516) and enters the inner pipe. It bypasses the locked coil (75) of the inner pipe and forms the take-up semi-cycle (82). Afterwards it comes out of the same lateral opening (516) of the inner pipe to form a sintered and unhitched hold-down mechanism at certain section of the stent. Passing through the inner layer between the inner pipe (51) and the medium pipe (88), it comes out of the near-end of the implantation system and is fixed temporarily to the near-end controller of the implantation system.

The fifth instance of the hold-down mechanism of the take-up wire in present invention is shown in FIG. 18. This hold-down mechanism of the take-up wire also has a structure of single take-up wire and multi-sections of single direction. A hold-down mechanism of the flexible connect collar is placed on the several compressed sections of the stent (1) (The structure of each hold-down mechanism of the flexible connect collar is identical with that of the third instance in FIG. 16.). The hold-down mechanism of the flexible connect collar on the same section wraps the stent under radial compression about one circle and forms two opposite free cycles (721, 722) near the propinquity (The two free cycles may not approach to each other and have a far distance, e.g. a half cycle apart or less). The same take-up wire (8) is locked by the locked coil at its far-end take-up collar (81). Then it passes the two opposite free cycles of the hold-down mechanism of the flexible connect collar on different sections and wraps the sintered and unhitched mechanism to form a continuous multi-sectional hold-down mechanism. The detailed process of wrapping is as follows:

The take-up wire (8) is locked by the locked coil (75) at its far-end take-up collar (81), and passes through a lateral opening (516) of the inner pipe. Afterwards it passes the first free cycle (721) on the first compressed section and curves to the second free cycle (722) on the same section. And then it passes through the second free cycle and enters the second compressed section of the stent (1). It curves back the first compressed section at the second free cycle (722) on the second compressed section and forms the take-up semi-cycle (82) at the turning. The take-up wire (8) passes through the second free cycle (722) on the first compressed section again and curves to the first free cycle (721) on the same section. And then it passes through the first free cycle (721) and enters the second compressed section of the stent (1) again. Afterwards it passes through the first free cycle (721) on the second compressed section and curves to the second free cycle (722) on the same section. Then it passes through the second free cycle (722) and the take-up semi-cycle (82) and enters the third compressed section of the stent (1) . . . . At last, it enters another lateral opening (516) of the inner pipe (51) and passes through the last take-up semi-cycle (82), which is formed by the same or the other locked coil (75) and is locked by the passing locked coil (75) temporarily. In the process of wrapping, the take-up wire forms a double line parallel segment between the two opposite free cycles at the same compressed section. It forms a double line vertical segment at one side of two compressed sections and a single line vertical segment at the other side of two compressed sections. The close segment of the take-up wire can pass the take-up space (52) of the inner pipe (51) or the inner layer between the inner pipe (51) and the medium pipe (88). And then it comes out the near-end of the implantation system and is fixed temporarily to the near-end controller of the implantation system.

The sixth instance of the hold-down mechanism of the take-up wire in present invention is showed in FIG. 19. This hold-down mechanism of the take-up wire also has a structure of single take-up wire and multi-sections of single direction. A closed line eye (103) or a closed flexible connect collar (7) is set up on several compressed sections of the stent (1). The closed line eye of each section is set up on the same lengthwise long line or different rotating angle of the stent. The same take-up wire (8) is locked by the locked coil (75) at its far-end take-up collar (81), and passes through the closed line eye (103) and a closed flexible connect collar (7) on different sections of the stent in succession to form a continuous multi-sectional hold-down mechanism. The detailed process of wrapping is as follows:

The take-up wire (8) is locked by the locked coil (75) at its far-end take-up collar (81), and passes through a lateral opening (516) of the inner pipe. Afterwards it passes the closed line eye (103) on the first compressed section and wraps the surface of the stent under radial compression widthwise about a circle. Then it passes through the closed flexible connect collar (7) which is set up on the same section. It passes the same closed line eye (103) again and enters the second compressed section of the stent. And then it wraps a take-up semi-cycle (82) at the closed line eye (103) of the second section and curves back the closed line eye (103) of the first section. Then it passes the closed line eye (103) and wraps the surface of the radially compressed stent widthwise about a circle. It passes the same closed line eye (103) and enters the second compressed section of the stent. And then it passes the closed line eye (103) of the second compressed section of the stent (1). And then it wraps the surface of the stent under radial compression widthwise about a circle. Then it passes through the same closed flexible connect collar (7) and the take-up semi-cycle (82) and enters the third compressed section of the stent (1) . . . . At last, it enters another lateral opening (516) of the inner pipe (51) and passes through the last take-up semi-cycle (82), which is formed by the same or the other locked coil (75) and is locked by the passing locked coil (75) temporarily. The close segment of the take-up wire can pass the take-up space (52) of the inner pipe (51) or the inner layer between the inner pipe (51) and the medium pipe (88). And then it comes out the near-end of the implantation system and is fixed temporarily to the near-end controller of the implantation system.

The hold-down mechanism of the take-up wire in present invention can also be single take-up wire and multi-sections of double directions, double take-up wires and multi-sections of single direction, double take-up wires and multi-sections of double directions, or the combination of the above single sectional and multi-sectional hold-down mechanisms. Because their detailed structures are similar to the above instances, no more instances are listed here.

The hold-down mechanism of the flexible connect collar in present invention also contains the temporary stayguy (4) which is applied to compose the implantation system. The hold-down mechanism of the flexible connect collar in the seventh instance, as shown in FIG. 20, owns such structures in present invention. The take-up wire (8) is locked by the locked coil (75) between the inner pipe (51) and the medium pipe (88) at its far-end take-up collar (81), and passes through the closed line eye (103) of the stent from inside to outside. And then it wraps the surface of the stent under radial compression widthwise about a circle and passes through the same closed line eye (103) from outside to inside. It enters the inner layer between the inner pipe (51) and the medium pipe (88). And then it comes out the near-end of the implantation system and is fixed temporarily to the near-end controller of the implantation system. The far-end of the temporary stayguy is set up between the inner pipe and the medium pipe. It bypasses the take-up (8) near the far-end take-up collar to form double lines and draws the take-up wire towards near-end.

The working principle of the hold-down mechanism of the take-up wire is as follows:

a) The take-up wire is a soft thin thread. Any line inflexion or take-up semi-cycle can be deformed and stretched. The position of the segments of the take-up wire of the stent can be changed.

b) The length of the double vertical segment which is formed in wrapping process of the take-up wire represents the degree of tightness and the degree of the radial compression of the stent. By means of drawing the near-end of the take-up, both sides of the same closed line eye through which the take-up passes or the both sides of the same closed free cycle or the two opposite free cycles are made to frap temporarily, compressing the inside stent radially.

c) Except for the thick texturing tube inside the inner pipe, lateral texturing tube is placed inside or on the inner pipe to lead threads to the direction vertical to the inner pipe. The location and rotating direction of the far-end opening of the lateral texturing tube maintains a standing relationship with the lateral opening of the inner pipe, hence having an indirect and adjustable relationship with the stent or self-expanding fenestrated covered stent. The stent has uncovered deformable unit and the fenestrated covered stent has a window through which threads can pass.

d) If the locked coil is pulled towards near-end, it will slide to the near-end and the far-end of the locked coil will slide out of the far-end take-up collar of the take-up wire or slide out of the last take-up semi-cycle. Then the take-up wire is separated and released from the locked coil.

e) When the take-up wire is released, drawing the near-end of the take-up can release all the connections of the take-up wire in opposite direction of sintering. All the take-up wire can be pulled to the near-end of the implantation system.

f) Different from the deformable unit of the stent, the size and shape of the closed line eye of the stent does not change with the diameter of the stent, even under the drawing of the passing take-up wire.

g) Temporary stayguy can hitch the take-up wire or the take-up collar with the assistance of the locked coil. If the stayguy is pulled towards the near-end, the far-end take-up is made to collar slide towards the near-end of the implantation system.

The process of usage, working principle and functions of the self-expanding stent in present can be elucidated generally as follows:

1. Assembly

The assembly of the self-expanding stent (1) and the delivery device (2) contains the preparation of the stayguy (70) of the inner pipe and temporary recycle wire (72); the passage of the stayguy (70) through the stent before operation; the adjustment of rotation in advance; the returning of the stayguy (70) to the near-end controller (80) with the assistance of temporary recycle wire (72). Two schemes can be selected to simplify this process to the lowest degree:
a. As shown in FIG. 5c, FIG. 5d, the locked coil (75) in the inner pipe (51) is in place. Every stayguy (70) on the lateral openings (516d, 516c, 516p) of the inner pipe has been locked by the locked coil (75). But the outside segment (703) of the stayguy is located outside the inner pipe. Temporary recycle wire (72) of the close segment of the stayguy is placed inside the inner pipe (51). The recycle collar (721) is placed outside the lateral openings (516d, 516c, 516p) of the inner pipe and the recycle wire (72) is located inside the inner pipe. The near-end (722) of the temporary recycle wire (72) expands to the near-end controller (80) and stretches out of special branch pipe of the stayguy (81). The stayguy (70) passes through a certain arched inflexion (102) or closed line eye (103) of the stent and through the recycle collar (721) of the recycle wire (72). After the near-end (722) of the recycle wire (72) is pulled, the near-end (708) of the stayguy will be recycled and drawn outside the special branch pipe of the stayguy (81) of the near-end controller (80).

Finally, the stayguy (70) will be replaced by recycle wire (72)
b. Similar to (a), but after the stayguy (70) passes through the arched inflexion (102) or closed line eye (103) of the stent, it knots with temporary recycle wire (72) to form a long stayguy (70). Every lateral opening (516d, 516c, 516p) of the distal segment (513) of the inner pipe is placed on the same reference plane (RP). With the assistance of the stayguy (70) and the locked coil (75), the stent is fastened and compressed radially to the inner pipe (51). The stayguy (70) passes out and in the deformable unit on the circumference of the stent. The relationship of the rotating angle between the stent and the lateral openings (516d, 516c, 516p) of the inner pipe or the reference plane (RP) can be determined. Half or whole circumference of the deformable unit can be used as unit-level to adjust and determine the rotating angle for the previous assembly in vitro. Every delivery device (2) has several following combining patterns to fix the far-end, medium section, near-end of the stayguy (70) to the inner pipe (51) of the delivery device (2): 1. single stayguy (70), single locked coil (75); 2. multi-stayguy (70), single locked coil (75); 3. two sets or more independent single locked coil (75) and relevant stayguy (70).

2. Radial Compression

The radial compression of the self-expanding stent contains the compression of the stent when it is frapped by the stayguy (70), the compression of entering an external protection apparatus (90, 92 and 96). That is, the stent under radial compression enters the outer sheath (90) or the tearable external protection apparatus (92) or the sintered and unhitched hold-down mechanism (96).

3. Entry

The entry of the self-expanding stent and the delivery device contains:

3.1 The preparation work of inserting: inserting a thread of 0.035″ into the left ventricle; inserting a thread of 0.014″ into the left or right coronary artery under the conditions of using the tearable external protection apparatus (92) or the sintered and unhitched hold-down mechanism (96) or the hold-down mechanism of the flexible connect collar or the hold-down mechanism of the take-up wire.

3.2 Relevant threads enter the relevant texturing tube (61, 99).

3.3 The delivery device (2) enters the blood vessel along the thread. The stent enters the blood vessel under the protection of the outer sheath (90) or the tearable external protection apparatus (92) or the sintered and unhitched hold-down mechanism (96) or the hold-down mechanism of the flexible connect collar or the hold-down mechanism of the take-up wire.

3.4 Before it enters the aortic arch, the outer sheath (90) stops moving until the compressed stent, the inner pipe (51) and the medium pipe come out.

3.5 The sliding between the inner pipe and the medium pipe.

3.6 The compressed stent passes through the aortic arch without outer sheath (90).

3.7 After the stayguy (70) is frapped, the stayguy (70) in the inner pipe (51) is drawn and shortened, while the inner pipe itself can not be compressed axially. The softer and thinner distal segment (513) and the medium segment (514) of the inner pipe of the delivery device (2) bend the straight inner pipe and increase the degree of curvature of the pre-made medium segment (514) of the arch-shape inner pipe, especially when the medium segment (514) of the inner pipe has been placed in the aortic arch. The distal segment (513) of the inner pipe is still a straight line when fastened by the compressed stent. The close segment (515) of the delivery device is still a straight line when fastened by the thicker and harder medium pipe.

3.8 Pulled simultaneously, the stayguy (70) and the locked coil (75) slide naturally to the concave plane (517) of the inner pipe, while the straight thread and texturing tube slide naturally to the convex plane (518) of the inner pipe.

4 The Location of the Self-Expanding Stent

The arched medium segment (514) of the inner pipe of the delivery device (2) constitutes a coincident or relevant reference plane (RP) to the plane of the aortic arch, having a fixed space rotating reference plane with two openings of coronary artery. The lateral texturing tube (99) of the delivery device (2) contributes to the location of the axial up and down section, rotating angle.

5 Expanding But not Releasing

When the stayguy (70) is loosened but the locked coil (75) does not slide, the self-expanding stent expands radially but is not released.

6 The Possibility of being Compressed after Expanding

After the stayguy (70) is frapped, the self-expanding stent will be compressed radially.

7 Releasing the Expansion

When the locked coil (75) slides towards the near-end, the relevant stayguy (701) is unlocked and the self-expanding stent will release the expansion. The process includes following steps: one-off releasing reaches radial expansion; stepped releasing arrives at radial expansion; releasing from the far-end of the up section to the medium section and to near-end of the down section reaches the expansion; releasing the outer circularity (155) of the medium section or the outer free tongue (156) of the outer section reaches the expansion; after location, releasing the far and near ends arrives at the expansion. Adjust the position when releasing the expansion.

8. The Possibility of Compressing Back the Outer Sheath (90), after the Radial Expansion and Before the Release of the Near-End

Before the release of the near-end in the stepped releasing, the self-expanding stent presents conical contour. The self-expanding stent can be compressed back to the outer sheath (90) again.

9. Fixation

Fix the self-expanding stent onto special position.

In conclusion, the delivery device of the self-expanding stent in present invention has the following features and merits:

1. Make the stent rotate and locate

The lateral openings (516d, 516c, 516p) of the distal segment of the inner pipe are placed at the same rotating angle. After the stayguy (70) is frapped, the medium section (514) of the inner pipe bends under the pull of the stayguy (70). The lateral openings (516d, 516c, 516p) of the distal segment of the inner pipe are located at the concave plane (517) of the bending inner pipe. The stayguy (70) and the locked coil (75) slide naturally to the concave plane (517) of the bending pipe. The 0.035″ texturing tube (61) or the thread space (53) slides naturally to the convex plane (518) of the bending pipe under the pull of the straight thread and distributes eccentrically. Especially when it passes through the aortic arch, the medium segment (514) of the inner pipe bends after being frapped by the stayguy. And the reference plane (RP) of the medium segment coincident with the aortic arch is determined. The two openings of the coronary artery (CA) and the lateral openings (516d, 516c, 516p) of the inner pipe or the reference plane (RP) exists rotary relationship. The stent can be fixed onto the lateral openings (516d, 516c, 516p) of the inner pipe. The rotary relationship between them can be adjusted previously in vitro by half deformable unit (101).

2. The locked coil (75) is installed. The locked coil (75) of the delivery device (2) can release the stent between two beats of the heart quickly and resistancelessly. One locked coil (75) can be released from far to near in order. Two or more locked coils (75) can be released selectively.

3. The tearable external protection apparatus (92) can be installed. The tearable sheath (94) and take-up wire (95) of the sheath are soft and thin in the tearable external protection apparatus (92). They can replace the harder outer sheath (90). The take-up wire (95) of the sheath can pass through the stent. On the same section in the middle of the stent, a lateral thread can pass through the lateral texturing tube (99).

4. The sintered and unhitched hold-down mechanism (96) and the hold-down mechanism of the flexible connect collar or the hold-down mechanism of the take-up wire can be installed. These mechanisms bend well and can replace the harder outer sheath (90) to frap the stent under radial compression.

5. B-mode ultrasonic probe (87) is installed. One or more B-mode ultrasonic probes (87) can be installed selectively on the distal segment (513) of the inner pipe.

6. The medium pipe (88) and the stayguy (89) of the medium pipe are installed. After the stayguy (89) is pulled, the strain increases and the medium pipe (88) bends, which assist the delivery device to pass the aortic arch. The medium pipe (88) slides along the inner pipe and the far-end (881) of the medium pipe can push the compressed stent out of the outer sheath (90).

7. The recycle wire (72) is installed in the close segment of the stayguy. Post-operative the recycle wire (72) can assist the stayguy (70) to come back the near-end controller (80).

### Industry Application

The delivery device of the self-expanding stent in present invention has the following merits and positive effects:

1. Rotary location of the stent; fixing the expanded stent effectively; reducing the valvular abrasion of artificial cardiac stent; reducing the abrasion of stayguy, avoiding the dislocation of stayguy.

2. The locked coil is installed to release the stent between two beats of the heart quickly and unresistedly.

3. The tearable external protection apparatus is installed to replace the harder outer sheath and to protect the implantation of the stent.

4. The sintered and unhitched hold-down mechanism is installed to replace the harder outer sheath and to protect the implantation of the stent.

5. B-mode ultrasonic probe is installed to monitor the process of the implantation of the stent.

6. The medium pipe and the stayguy of the medium pipe are installed to assist the delivery device to pass the aortic arch.

7. The temporary recycle wire is installed in the close segment of the stayguy to assist the stayguy to come back to the near-end controller before operation.

8. The merits and features of the hold-down mechanism used as external protection apparatus:

a) Adopting the hold-down mechanism of the take-up wire can make the stent under radial compression and the implantation system very soft and bend well particularly. And the compressed stent can be implanted to the far and bending blood vessel.

b) The hold-down mechanism of the take-up wire has a thin thickness and decreases the section of the whole stent under radial compression and the implantation system. When the soft thread with 0.05 mm diameter is used, its thickness decreases a lot compared with that of ordinary outer sheath with 0.20-0.30 mm thickness. The soft thread is made up of PTFE fiber, ePTFE fiber, Dacron fiber and is of high strength. Under the pressing of the external force, the line of the circum-section can be squashed and the whole diameter is reduced further.

c) After the hold-down mechanism of the take-up wire is released, the radial compression of the stent has no or little friction.

d) Regarding the double layer stent with outer free tongue, the outer tongue can expand under the condition that the inner layer of the stent is unexpanded. The outer free tongue plays a role of reference, and assists the location of the radially compressed stent in up and down axial direction and the rotating direction around the axis of the stent.

e) The take-up collar and the closed line eye of the stent can be located at different level. When the take-up wire goes outside of the inner pipe, the structure of the inner pipe is simplified and the diameter of the inner pipe and the compressed stent is decreased. The level positions of the take-up collar and the closed line eye have a certain distance towards the near-end, which fit the system into the stent with different length.

f) After the far-end take-up collar or the take-up semi-cycle of the take-up wire is locked twice by the locked coil, the medium and closed segments of the take-up wire can go inside the inner pipe, which simplifies the structure and decreases the diameter of the inner pipe.

g) The hold-down mechanism of the take-up wire of the self-expanding stent in present invention is an open system under radial compression. The lateral thread can pass through the releasing system. The lateral texturing tube can be fixed onto the inner pipe of the stent. When the stent is compressed radially, the thread can pass through any angle of the open structure of the stent under radial compression. And then the thread passes through the stent and enters the lateral texturing tube under the stent. The lateral thread can enters the branch of the blood vessel in advance and plays a role of reference assisting the location of the stent under radial compression in up and down axial direction and the rotating direction around the axis of the stent.

h) The hold-down mechanism of the take-up wire of the self-expanding stent in present invention has an open structure. After the stent expands and adheres to the pipe wall of the blood vessel, the blood flows to the branch of the blood vessel and passes the opening of the stent.

i) The hold-down mechanism of the take-up wire of the self-expanding stent in present invention is not influenced by the change of the length in the process of the radial compression and expansion of the stent. In the process of the radial compression, every closed line eye or free cycle of the flexible connect collar passed by the take-up wire is independent and is not influenced by the change of the length of the stent.

j) The take-up wire sintered the closed line eye or free cycle can only slide on the outer surface of the stent with minor magnitude. The hold-down mechanism of the take-up wire can be assembled before operation. During the operation, the doctor can compress the self-expanding stent or stent radially by frapping the take-up wire.

k) With the assistance of the closed line eye or free cycle, the hold-down mechanism of the take-up wire can wrap the stent outside near 360° or only a small radian. Because the take-up wire of the stent can compress the stent radially by wrapping a small part of the circumference of the stent only, it can be recycled conveniently.

m) Each medium and close segment of the take-up wire can go inside the take-up space of the locked coil of the inner pipe or between the inner pipe and stent (outside the inner pipe but inside the stent) or outside the stent, and do not interfere with each other. If the take-up wire can go outside the take-up space of the locked coil of the inner pipe, the interference between the take-up wire and the locked coil can be avoided, the space of the take-up space of the locked coil of the inner pipe can be saved and the flow of assembly can be simplified.

9. The merits and features of the hold-down mechanism of the flexible connect collar used as external protection apparatus:

a) The structure of the hold-down mechanism of the flexible connect collar is simple. The stent under radial compression and the implantation system are very soft and bend well particularly. And the compressed stent can be implanted onto the far and bending blood vessel.

b) The hold-down mechanism of the flexible connect collar has a thin thickness, which decreases the section of the whole stent under radial compression and the implantation system. When the soft thread with 0.05 mm diameter is used, its thickness decreases a lot compared with that of common outer sheath with 0.20-0.30 mm thickness. The soft thread used by the flexible connect collar is made up of DACRON fiber, PA fiber, POLYETHYLENE fiber, POLYPROPYLENE fiber and is of high strength. Under external force, the line of the circum-section can be squashed and the whole diameter is decreased further.

c) After the hold-down mechanism of the flexible connect collar is released, no or little friction exists in the process of radial expansion of the stent.

d) The hold-down mechanism of the flexible connect collar of the self-expanding stent in present invention is not only an open system under radial compression but a releasing system. The lateral texturing tube and its opening can be fixed to the inner pipe of the stent. When the stent is compressed radially, the thread can pass through any angle of the open structure of the stent under radial compression. And then the thread passes through the stent and enters the lateral texturing tube under the stent. The lateral thread enters the branch of the blood vessel in advance and plays a role of reference assisting the location of the stent under radial compression in up and down axial direction and the rotating direction around the axis of the stent.

e) The hold-down mechanism of the flexible connect collar of the self-expanding stent in present invention has an open structure. After the stent expands and adheres to the pipe wall of the blood vessel, the blood flows to the branch of the blood vessel and passes the opening of the stent.

f) The hold-down mechanism of the flexible connect collar of the self-expanding stent in present invention is not influenced by the change of the length in the process of the radial compression and expansion of the stent.

g) To the covered stent, the free cycle of the flexible connect collar outside the expanding covered stent clips between the stent and the pipe wall of the blood vessel to prevent the blood flowing between the stent and the pipe wall of the blood vessel.

1. A delivery device for delivering a self-expanding stent comprising
a pipe head, an inner pipe, a near-end controller, a medium pipe, a texturing tube, an external protection apparatus, at least one locked coil and one stayguy;
said pipe head, inner pipe and near-end controller being interconnected in order and communicated each other;
a sleeve pipe of the medium pipe sliding along the inner pipe;
said texturing tube being installed in an incorporated body of the pipe head, inner pipe and near-end controller;
said external protection system enveloping the outside of the inner and medium pipes;
said locked coil and stayguy thread going through the inner pipe and a near-end controller, which are incorporated together.

2. The delivery device of claim 1, wherein the inner pipes is a long tube structure having at least one pore space for passing various strings and threads through in the inner pipe and at least one lateral opening is installed in the far-end of the inner pipe.

3. The delivery device of claim 2, wherein at least one texturing tube, through which various strings and threads pass, is installed in the inner pipe, each texturing pipe is able to slide with the other and with the inner pipe.

4. The delivery device of claim 2, wherein at least one texturing tube, through which various strings and threads pass, is installed in the inner pipe, the texturing pipes are set to affix with other texturing pipes and inner pipes and cannot slide along each other.

5. The delivery device of claim 2, wherein a braided strengthening net can be installed in the intermediate layer of the inner pipe, a lateral opening of the inner pipe can be located in one of meshes of the braided strengthening net.

6. (canceled)

7. (canceled)

8. (canceled)

9. The delivery device of claim 1, wherein a close segment of the medium pipe connects with a lateral pipe, one stayguy is set up in the medium pipe, the far-end of the stayguy is fixed on a far-end port of the medium pipe, a near-end of the stayguy is drawn out from the lateral pipe of the close segment of the medium pipe, a fixed point of the far-end of the stayguy and the lateral pipe of the close segment of the medium pipe are set up at a same plane, side each port of the near-end of the medium pipe and each port of the lateral pipe have a shrink-ring, which can slide along the inner pipe when it is loosened and be fixed on certain position of the inner pipe when tightened.

10. (canceled)

11. The delivery device of claim 1, wherein the external protection apparatus is a take-up and hold-down mechanism, which contains at least one locked coil, at least one connect collar linking to the stent, at least one take-up wire used for compressing the stent, a take-up collar is set up on a far-end of the take-up wire, after it is locked at its far-end by the take-up collar, the take-up wire passes through the lateral opening of the inner pipe and wraps between the connect collar on the locked coil, stent and the outboard stent, forming a hold-down mechanism which can be locked or unlocked as the case might be, the near-end of the take-up wire is drawn out of the near-end of the implantation system and is fixed onto the near-end controller of the implantation system temporarily.

12. The delivery device of claim 11, wherein one take-up wire and one connect collar are installed, the connect collar is a closed line eye or a closed flexible connect collar linking to the stent, locked at its far-end cycle by the locked coil, the take-up wire passes through the closed line eye or the closed flexible connect collar on the same section of the stent and rounds the locked coil in the inner pipe to form a semi-cycle of take-up wire, in the end, it circles outside the stent once and wraps to form a hold-down mechanism which can be locked or unlocked as it depends at a certain section of the stent.

13. The delivery device of claim 11, wherein one take-up wire and one connect collar are installed. The connect collar is a closed line eye or a closed flexible connect collar linking to the stent, locked at its far-end cycle by the locked coil, the take-up wire passes through the closed line eye or the closed flexible connect collar on the same section of the stent and rounds the locked coil in the inner pipe to form a semi-cycle of take-up wire. In the end, it circles outside the stent twice in both directions and wraps to form a hold-down mechanism which can be locked or unlocked as it might be at a certain section of the stent.

14. The delivery device of claim 11, wherein one take-up wire and two connect collars are installed, the two connect collars are placed by a hold-down mechanism of flexible connect collar on the stent, the hold-down mechanism of flexible connect collar circles the stent under radial compression in less than one lap, forming two opposite free cycles as the connect collars. Locked at its far-end cycle by the locked coil, the take-up wire passes through the two free cycles of the hold-down mechanism of the flexible connect collar and rounds the locked coil in the inner pipe to form a semi-cycle of the take-up wire, forming a hold-down mechanism which can be locked or unlocked as it depends at a certain section of the stent.

15. The delivery device of claim 11, wherein one take-up wire and several connect collars are installed, several connect collars are provided by a hold-down mechanism of flexible connect collar on the stent, the hold-down mechanism of flexible connect collar circles the stent under radial compression in less than one lap, forming two opposite free cycles as the connect collars. Locked at its far-end cycle by the locked coil, the take-up wire passes through two opposite free cycles of the hold-down mechanism of the closed line eye or the flexible connect collar at different sections of the stent one after another and carries out lockable and undone circles, forming a continuous multi-sectional hold-down mechanism.

16. The delivery device of claim 11, wherein one take-up wire and several connect collars are installed, several connect collars are provided by a hold-down mechanism of flexible connect collar on the multi-sectional stent, every hold-down mechanism of the flexible connect collar circles the stent under radial compression in less than one lap, forming two opposite free cycles as the connect collars, locked at its far-end cycle by the locked coil, the take-up wire wraps the stent at different lockable and undone sections in one direction and comes back along the same course to warp the lockable and undone stent for the second time, forming a continuous multi-sectional hold-down mechanism. In the process of the winding, the take-up wire rounds the locked coil in the inner pipe to form a semi-cycle of the take-up wire.

17. The delivery device of claim 11, wherein two take-up wires and several connect collars are installed, several connect collars are provided by a hold-down mechanism of the flexible connect collar on the multi-sectional stent, every hold-down mechanism of the flexible connect collar circles the stent under radial compression in less than one lap, forming two opposite free cycles as the connect collars, locked at their far-end cycles by the locked coil, the two take-up wires wrap the stent at different lockable and undone sections in opposite symmetrical directions, forming a continuous multi-sectional hold-down mechanism. In the process of the winding, two take-up wires round the locked coil in the inner pipe, forming two semi-cycles of the take-up wires, respectively.

18. The delivery device of claim 11, wherein two take-up wires and several connect collars are installed, several connect collars are provided by a hold-down mechanism of the flexible connect collar on the multi-sectional stent, every hold-down mechanism of the flexible connect collar circles the stent under radial compression in less than one lap, forming two opposite free cycles as the connect collars, locked at their far-end cycles by the locked coil, the two take-up wires wrap the stent at different sections lockably and unlockably in one of the opposite symmetrical directions and come back along the same course to wrap the stent lockably and unlockably for the second time. In the end, they form a continuous multi-sectional hold-down mechanism. In the process of the winding, two take-up wires round the locked coil in the inner pipe to form semi-cycles of the take-up wire, respectively.

19. The delivery device of the self-expanding stent of claim 14, wherein the hold-down mechanism of the flexible connect collar is a flexible connect collar with two free cycles whose fixed point circles and ties to a cross-point of mesh wires, two free cycles extend bilaterally along the outside superficial circularity of the stent and circle the stent under radial compression in less than one lap to form an opposite condition.

20. The delivery device of the self-expanding stent of claim 14 wherein the hold-down mechanism of the flexible connect collar contains several flexible connect collars, the fixed points of two flexible connect collars among them circle and tie to the same cross-point of the mesh wires, the fixed points of the other flexible connect collars circle and tie to other cross-points of the mesh wires in the same circumference of the above cross-point, the free cycle of every flexible connect collar extends bilaterally along the outside superficial circularity of the stent and circle the stent under radial compression in less than one lap, extending in the same direction, the free cycles are connected one by one in order, extending in two directions, the last two free cycles form an opposite condition.

21. The delivery device of claim 11, wherein the hold-down and take-up mechanism also contains temporary stayguys, locked temporarily at its far-end cycle by the locked coil, the take-up wire passes through a closed line eye on the stent and then circles the surface of the stent under radial compression close to a cycle and comes back to the same closed line eye again, afterwards, it passes through the interlayer between the inner pipe and the medium pipe and is drawn out of the near-end of the implantation system, the temporary stayguy is set between the inner pipe and the medium pipe, the temporary stayguy rounds the take-up wire near the far-end of its cycle to form double lines and holds the take-up wire to the near-end.

22. The delivery device of claim 1, wherein the external protection apparatus is a hold-down mechanism of the flexible connect collar, which contains at least one locked coil, one flexible connect collar linked to the stent and applied to compress the stent, one flexible connect collar passed through the lateral opening of the inner pipe and locked by the temporarily passing locked coil.

23. The delivery device of claim 22, wherein the flexible connect collar contains fixed ends and free ends, the fixed point circles and ties to the arched inflexion or the closed line eye or the cross-point of the mesh wires of the stent, the free point extends inside or outside the stent and composes the single free cycle or double free cycle.

24. The delivery device of claim 22, wherein the flexible connect collar is a single closed wire loop, which passes through the arched inflexion or the closed line eye or the cross-point of the mesh wires of the stent. It can slide but cannot break away.

25. The delivery device of claim 22, wherein the hold-down mechanism of the flexible connect collar contains several flexible connect collars with a single free cycle, the flexible connect collars are distributed circularly on the same section inside or outside the stent, the adjacent flexible connect collars under the radial compressed stent are connected one by one in order and circle the stent once, the last free cycle of the flexible connect collar passes through the lateral opening of the inner pipe and is locked by the temporarily passing locked coil.

26. The delivery device of claim 22, wherein the hold-down mechanism of the flexible connect collar contains only one flexible connect collar with a single free cycle, the flexible connect collar under the radial compressed stent circles outside the stent once, passes through the lateral opening of the inner pipe, and then is locked by the temporarily passing locked coil.

27. The delivery device of claim 22, wherein the hold-down mechanism of the flexible connect collar contains only one flexible connect collar with a double free cycle, the two free cycles of the flexible connect collar circle the radial compressed stent in opposite directions half cycle, respectively, they pass through the same lateral opening of the inner pipe and are locked by the temporarily passing locked coil.

28. The delivery device of claim 22, wherein the hold-down mechanism of the flexible connect collar contains several flexible connect collars with a single free cycle, the flexible connect collars are distributed helically outside the stent, the adjacent flexible connect collars under the radial compression of the stent are connected one by one in order and circle the stent at least once, the last free cycle of the flexible connect collar passes through the lateral opening of the inner pipe and is locked by the temporarily passing locked coil.

29. The delivery device of claim 22, wherein the hold-down mechanism of the flexible connect collar contains one flexible connect collar with a double free cycle and several flexible connect collars with a single free cycle, the flexible connect collars are distributed helically outside the stent, one flexible connect collar with a double free cycle is set up in the middle of the stent, two free cycles of the flexible connect collar extend in opposite directions under the radial compression of the stent and are connected with the adjacent flexible connect collar with a single free cycle one by one in order at least once, the last two free cycles extending in opposite directions of the flexible connect collar pass through the far lateral opening and the proximal opening of the inner pipe respectively and are locked by the temporarily passing locked coil.

30. The delivery device of claim 22, wherein the hold-down mechanism of the flexible connect collar also contains temporary stayguys which make up of the implantation system, the flexible connect collar applied to compress the stent is a single closed wire loop, one port passes through the arched inflexion or the closed line eye or the cross-point of the mesh wires of the stent, another one passes through the lateral opening of the stent and the inner pipe to enter the inner pipe, or enters between the inner pipe and the medium pipe and is locked by the temporarily passing locked coil, the middle one is pulled by the temporary stayguys.

31. The delivery device of claim 30, wherein the closed wire loop is passed by a stayguy and can be pulled to the near-end, a cycle is set up at the far-end of the stayguy and is locked by the temporarily passing locked coil of the inner pipe or the locked coil between the inner pipe and the medium pipe.

32. The delivery device of claim 1, wherein at least one lateral texturing tube is installed, beginning from the outside of the medium section to the distal segment of the inner pipe, the lateral texturing tube is connected with the distal segment of the inner pipe, extending to the medium segment of the inner pipe along the proximal direction of the inner pipe, the lateral texturing tube can also extend to the close segment of the inner pipe or near-end controller, located between the far lateral opening and the proximal opening of the inner pipe, the far-end of the lateral texturing tube bends outward to form a certain angle between the direction of the port and the direction of the lateral opening of the inner pipe.

33. (canceled)

34. (canceled)

35. The delivery device of claim 1, wherein the locked coil passes through the inner pipe, its far-end passes through the cycle of one or more stayguys and locks one or more stayguys, its near-end is connected with the sliding-jig in the branch pipe of the locked coil of the near-end controller.

36. The delivery device of claim 1, wherein the stayguy passes through the pore space of the inner pipe, its far-end has a cycle of stayguy and its near-end is drawn out of the branch pipe of the stayguy of the near-end controller, its distal segment is drawn out of the lateral opening of the inner pipe, forming the outside segment of the stayguy, the outside segment of the stayguy circles the net stent once, enters the same lateral opening of the inner pipe and then is locked through its far-end cycle of the stayguy by the locked coil.

37. The delivery device of claim 36, wherein the outside segment of the stayguy passes through the opening of the deformable unit or the arched inflexion or the closed line eye and the flexible connect collar to form a lasso when it circles the stent.

38. (canceled)

39. The delivery device of claim 1, wherein the temporary recovering wire is applied to assist the stayguy and comes back to the near-end controller when the device is assembled.
